(19) United States
(12) Patent Application Publication
(10) Pub. No.: US 2025/0360227 A1
(43) Pub. Date: Nov. 27, 2025

(54) MELANOPSIN VARIANTS FOR VISION RESTORATION

(71) Applicant: Adverum Biotechnologies, Inc., Redwood City, CA (US)

(72) Inventor: Cameron BAKER, San Francisco, CA (US)

(21) Appl. No.: 18/872,410

(22) PCT Filed: Jun. 6, 2023
(86) PCT No.: PCT/US2023/067969

§ 371 Date: Dec. 6, 2024

Related U.S. Application Data
(60) Provisional application No. 63/349,970, filed on Jun. 7, 2022.
(60) Provisional application No. 63/411,523, filed on Sep. 29, 2022.
(60) Provisional application No. 63/466,181, filed on May 12, 2023.

(51) Int. Cl. A61K 48/00; A61K 9/00; A61K 38/00; A61P 27/02; C07K 14/005; C07K 14/72; C12N 15/86

(52) U.S. Cl. CPC A61K 48/0058; A61K 9/0048; A61K 48/0075; A61P 27/02; C07K 14/005; C07K 14/723; C12N 15/86; A61K 38/00; C07K 2319/00; C12N 2750/14143; C12N 2750/14151

(57) ABSTRACT

Provided herein, inter alia, are melanopsin variants that demonstrate greater amplitude/conductance and/or faster off kinetics than the amplitude/conductance and/or the off kinetics of the wild type human melanopsin. Also provided are related nucleic acids, virions, host cells, methods of producing recombinant virions, and pharmaceutical compositions. Further provided are methods of using such melanopsin variants to restore or enhance visual function in a subject.

### CROSS-REFERENCE TO RELATED APPLICATIONS

This application is a national stage application under 35 U.S.C. § 371 of International Application No. PCT/US2023/067969, filed internationally on Jun. 6, 2023, which claims the priority benefits of U.S. Provisional Application 63/349,970, filed Jun. 7, 2022; U.S. Provisional Application 63/411,523, filed Sep. 29, 2022; and United States Provisional Application 63/466,181, filed May 12, 2023, the content of each of which is incorporated herein by reference in their entirety.

### REFERENCE TO AN ELECTRONIC SEQUENCE LISTING

The contents of the electronic sequence listing (627002001400seqlist.xml; Size: 162,974 bytes; and Date of Creation: Nov. 22, 2024) is herein incorporated by reference in its entirety.

### FIELD

The present application relates to melanopsin variants and uses thereof to restore or enhance visual function in a subject with impaired vision due to photoreceptor loss.

### BACKGROUND

The degeneration of light-detecting rod and cone photoreceptors in the human retina (e.g., due to disease, infection, or injury) typically leads to severe visual impairment and, in some cases, legal blindness in millions of people worldwide (McClements et al. (2020) Front Neurosci. 14:57090). However, while the causes of retinal degeneration may vary, there are considerable similarities in the physiological changes that occur in the retina. When patients suffer loss of photoreceptor cells but maintain remaining layers of cells in the neural retina, it may be possible to restore vision through optogenetic therapy, i.e., the provision of light-sensitive molecules to surviving cell types of the retina that enable light perception through the residual neurons. Current optogenetic approaches have been limited by low light sensitivity, slow kinetics, and/or narrow spectral response. Additionally, current approaches lack adaptation to changes in ambient light. Thus, there is a need in the art for improved optogenetic approaches for treatment of vision loss due to rod and cone degeneration.

### SUMMARY

In some embodiments, provided is a melanopsin variant comprising no more than amino acids 1-425 of a wild type human melanopsin set forth in SEQ ID NO: 1, wherein amplitude/conductance and/or off kinetics of the melanopsin variant are greater than the amplitude/conductance and/or faster than the off kinetics of the wild type human melanopsin. In some embodiments, the melanopsin variant comprises a sequence set forth in any one of SEQ ID NOs: 2, 3, 4, 82, 83, and 84 or a variant thereof that comprises one or more amino acid substitutions. In some embodiments, the melanopsin variant comprises one or more amino acid substitution(s) at P10, T83, T129, Q135, S183, Y212, E215, M226, Y382, S384, R386, and/or R390, wherein amino acid position(s) are relative to the wild type human melanopsin set forth in SEQ ID NO: 1. In some embodiments, the one or more substitution mutation(s) are selected from the group consisting of: P10F, T83L, T129S, Q135N, S183A, Y212F or Y212A, E215S, M226S or M226T, Y382E or Y382D, S384D, R386A, and R390A or R390D.

In some embodiments, the melanopsin variant comprises the P10F substitution. In some embodiments, the melanopsin variant comprises the sequence set forth in SEQ ID NO: 5. In some embodiments, the melanopsin variant comprises (such as further comprises) the T83L substitution. In some embodiments, the melanopsin variant comprises the sequence set forth in SEQ ID NO: 6. In some embodiments, the melanopsin variant comprises (such as further comprises) the T129S substitution. In some embodiments, the melanopsin variant comprises the sequence set forth in SEQ ID NO: 7. In some embodiments, the melanopsin variant comprises (such as further comprises) the Q135N substitution. In some embodiments, the melanopsin variant comprises the sequence set forth in SEQ ID NO: 8 or 9. In some embodiments, the melanopsin variant comprises (such as further comprises) the S183A substitution. In some embodiments, the melanopsin variant comprises any one of SEQ ID NOs: 10-12. In some embodiments, the melanopsin variant comprises (such as further comprises) the Y212F substitution. In some embodiments, the melanopsin variant comprises the sequence set forth in SEQ ID NO: 13. In some embodiments, the melanopsin variant comprises (such as further comprises) the M226S or the M226T substitution. In some embodiments, the melanopsin variant comprises the sequence set forth in SEQ ID NO: 14 or 15. In some embodiments, the melanopsin variant comprises (such as further comprises) the Y382E or the Y382D substitution. In some embodiments, the melanopsin variant comprises the sequence set forth in SEQ ID NO: 16. In some embodiments, the melanopsin variant comprises (such as further comprises) the S384D substitution. In some embodiments, the melanopsin variant comprises the sequence set forth in SEQ ID NO: 17. In some embodiments, the melanopsin variant comprises (such as further comprises) the R386A substitution. In some embodiments, the melanopsin variant comprises the sequence set forth in SEQ ID NO: 18 or 19. In some embodiments, the melanopsin variant comprises (such as further comprises) the R390A or the R390D substitution. In some embodiments, the melanopsin variant comprises the sequence set forth in any one of SEQ ID NOs: 20-23.

In some embodiments, provided is a melanopsin variant comprising at least amino acids 1-377 of a wild type human melanopsin set forth in SEQ ID NO: 1 fused to a c-terminal domain (CTD) of a heterologous G protein-coupled receptor (GPCR) or a CTD variant thereof, wherein amplitude/conductance and/or off kinetics of the melanopsin variant are greater than the amplitude/conductance and/or faster than the off kinetics of the wild type human melanopsin. In some embodiments, the CTD of the heterologous GPCR or CTD variant thereof is the CTD of a visual opsin or variant thereof. In some embodiments, the CTD of the visual opsin or variant thereof is a CTD of (i) a wild type D. melanogaster rhodopsin 1, (ii) a wild type human rhodopsin, (iii) a wild type human short wavelength opsin (hOPNISW), (iv) a wild type human medium wavelength opsin, or (v) a wild type human long wavelength opsin. In some embodiments, the CTD of the visual opsin or variant thereof comprises an amino acid sequence set forth in any one of SEQ ID NOs: 27-30 and 32-33. In some embodiments, the melanopsin variant comprises an amino acid sequence set forth in any one of SEQ ID NOs: 39-42, 44-45, and 63-65. In some embodiments, the melanopsin variant further comprises one or more substitution mutation(s) at P10, T83, T129, Q135, S183, Y212, E215, M226, Y382, S384, R386, R390, wherein amino acid position(s) are relative to the wild type human melanopsin set forth in SEQ ID NO: 1, wherein amino acid position(s) are relative to the wild type human melanopsin set forth in SEQ ID NO: 1. In some embodiments, the one or more substitution mutation(s) are selected from the group consisting of: P10F, T83L, T129S, Q135N, S183A, Y212F or Y212A, E215S, M226S or M226T, Y382E or Y382D, S384D, R386A, and R390A or R390D. In some embodiments, the melanopsin variant further comprises one or more amino acid substitutions in the CTD of the heterologous GPCR or the CTD variant thereof.

In some embodiments, provided is a melanopsin variant comprising one or more amino acid substitutions at P10, T83, T129, Q135, S183, Y212, E215, M226, Y382, S384, R386, and R390, wherein amino acid positions are relative to a wild type human melanopsin set forth in SEQ ID NO: 1, and wherein amplitude/conductance and/or off kinetics of the melanopsin variant are greater than the amplitude/conductance and/or faster the off kinetics of the wild type human melanopsin. In some embodiments, the one or more substitution mutation(s) are selected from the group consisting of: P10F, T83L, T129S, Q135N, S183A, Y212F or Y212A, E215S, M226S or M226T, Y382E or Y382D, S384D, R386A, R390A or R390D.

In some embodiments, the melanopsin variant comprises, for example, at least about any one of 80%, 85%, 90%, 91%, 92%, 93%, 94%, 95%, 96%, 97%, 98%, 99% overall sequence homology or identity to a melanopsin variant set forth in any one of SEQ ID NOs: 2-4, 5-23, 39-42, 44-45, 63-65, and 82-84. In some embodiments, the melanopsin variant has at least about 90% overall sequence homology or identity to a melanopsin variant set forth in any one of SEQ ID NOs: 2-4, 5-23, 39-42, 44-45, 63-65, and 82-84. In some embodiments, the melanopsin variant has at least about 95%, at least about 98%, at least about 99%, at least about 99.5%, or at least about 99.9% overall sequence homology or identity to a melanopsin variant set forth in any one of SEQ ID NOs: 2-4, 5-23, 39-42, 44-45, 63-65, and 82-84.

In some embodiments, the amplitude/conductance of a melanopsin variant provided herein is at least 1.25-fold greater than the amplitude/conductance of the wild type human melanopsin in HEK293T cells. In some embodiments, the off kinetics of a melanopsin described herein are at least 1.10-fold faster than the off kinetics of the wild type human melanopsin in HEK293T cells.

Also provided herein is a nucleic acid comprising a polynucleotide sequence that encodes the melanopsin variant provided herein. In some embodiments, the nucleic acid is operable linked to a promoter. In some embodiments, the promoter is a retinal cell-specific promoter. In some embodiments, the retinal cell-specific promoter is selected from the group consisting of: human synapsin (hSyn), SNCG, NEFH, NEFL, 4×grm6, and grm6. In some embodiments, the nucleic acid further comprises one or more enhancer sequences, intron sequences, leader sequences, Kozak sequences, poly A sequences, stuffer sequences, and/or inverted terminal repeat (ITR) sequences.

Also provided herein is a recombinant virion comprising: (a) a capsid protein and (b) the nucleic acid provided herein. In some embodiments, the capsid protein is selected from: AAV2-7m8, AAV2, AAV2-4YF, AAV9, AAV9-7m8, R100 and LSV1.

In some embodiments, provided is a host cell comprising a nucleic acid described herein. In some embodiments, the host cell further comprises one or more of: (1) a polynucleotide encoding a capsid protein; (ii) a polynucleotide encoding a rep protein; and (iii) AAV helper functions. In some embodiments, provided is a method for producing a recombinant virion, comprising: (a) culturing the host cell of claim 47 under conditions to produce the recombinant virion, and (b) recovering the recombinant virion produced by the host cell. In some embodiments, the method further comprises the step of purifying the recombinant virion.

Also provided herein is a pharmaceutical composition comprising the recombinant virion described herein and a pharmaceutically acceptable excipient. In some embodiments, provided is a method of restoring or enhancing visual function in a subject, comprising administering the pharmaceutical composition provided herein to the eye of the subject. In some embodiments, the administration comprises an intraocular injection, a subretinal injection, a suprachoroidal injection, or an intravitreal injection. In some embodiments, the subject has an ocular disease or disorder selected from the group consisting of: retinitis pigmentosa, macular degeneration, retinoschisis, Leber's Congenital Amaurosis, diabetic retinopathy, geographic atrophy, choroideremia, cone dystrophy, and cone-rod dystrophy. In some embodiments, the subject has experienced retinal detachment or photoreceptor loss due to ocular disease, infection, trauma, injury, impact to the head, acute light damage, UV light damage, laser damage, or chemical damage. In some embodiments, the subject is human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF DESCRIPTION OF THE DRAWINGS

FIG. 1 provides a schematic of wild-type (“WT”) human melanopsin (SEQ ID NO: 1), a seven transmembrane G-protein coupled receptor (GPCR) with a cytoplasmic tail. A putative protein kinase A (PKA) site, a putative a putative protein kinase C (PKC) site, and a putative phosphorylation and/or G protein-coupled receptor kinase (GRK)/arrestin binding are indicated.

FIG. 2 shows the results of experiments that were performed to determine the amplitude of calcium light responses from melanopsin truncation variants.

FIG. 3 shows the results of experiments that were performed to determine the TauOFF of calcium light responses from melanopsin truncation variants.

FIG. 4 shows a comparison of the amplitude versus the TauOFF for the truncation variants. Variants (black circles) like 419AA have both a smaller TauOFF and greater amplitude than wild type (white circle).

FIG. 5 shows the results of experiments that were performed to determine the amplitude of calcium light responses from melanopsin chimeric variants.

FIG. 6 shows the results of experiments that were performed to determine the TauOFF of calcium light responses from chimeric melanopsin variants.

FIG. 7 shows the results of experiments that were performed to determine the TauOFF of calcium light responses from the fastest chimeric melanopsin variants shown in FIG. 6. Error bars=SEM.

FIG. 8 shows a comparison of the amplitude versus the TauOFF for the fastest chimeric melanopsin variants shown in FIG. 7. Variants are black circles and wild type is a white circle.

FIG. 9 shows the results of experiments that were performed to determine the amplitude of calcium light responses from substituted full length melanopsin variants.

FIG. 10 shows the results of experiments that were performed to determine the TauOFF of calcium light responses from substituted full length melanopsin variants.

FIG. 11 shows the results of experiments that were performed to determine the TauOFF of calcium light responses from the fastest substituted full length melanopsin variants shown in FIG. 10. Error bars=SEM.

FIG. 12 shows a comparison of the amplitude versus the TauOFF for the fastest substituted full length melanopsin variants FIG. 10. Variants are black circles and wild type is a white circle. Error bars=SEM.

FIG. 13 shows the results of experiments that were performed to determine amplitude of calcium light responses from substituted melanopsin variants in a truncated backbone (419AA, SEQ ID NO: 3).

FIG. 14 shows the results of experiments that were performed to determine the TauOFF of calcium light responses from substituted melanopsin variants in a truncated backbone (419 AA, SEQ ID NO: 3).

FIG. 15 shows the results of experiments that were performed to determine the TauOFF of calcium light responses from the fastest substituted melanopsin variants in a truncated backbone shown in FIG. 14.

FIG. 16 shows a comparison of the amplitude versus the TauOFF for the fastest substituted melanopsin variants in a truncated backbone shown in FIG. 14. Variants are black circles and wild type is a white circle. Error bars=SEM.

FIG. 17 shows a schematic of a development strategy for designing melanopsin variants

FIG. 18 shows the results of experiments that were performed to determine the amplitude of calcium light responses for WT human melanopsin, and melanopsin variants 405AA, 425AA, V370-R377Del, and K356-R377Del over time.

FIG. 19 shows the results of experiments that were performed to determine the TauOFF of calcium light responses for WT human melanopsin and melanopsin variants 405AA, 425AA, V370-R377Del, and K356-R377Del over time.

FIG. 20 shows the results of experiments that were performed to determine the amplitude of calcium light responses from cells transduced with AAV2.7m8 carrying WT human melanopsin or a melanopsin variant comprising SEQ ID NO: 19.

FIG. 21 shows the results of experiments that were performed to determine the amplitude of calcium light responses of HEK293T cells transduced with different MOIs of AAV2.7m8-CMV-SEQ ID NO: 19.

### DETAILED DESCRIPTION

### Overview

One of the goals of optogenetic therapy is to provide the expression of light-sensitive proteins, i.e., opsins, in the cells of a damaged or degenerated retina. However, the low light sensitivity and the slow (seconds) kinetics of many opsins exclude them from practical use in the treatment of vision loss. Described herein are melanopsin variants that exhibit, e.g., greater amplitude/conductance (such as amplitude/conductance light responses) and/or faster OFF kinetics (e.g., faster OFF light response) than wild type human melanopsin. Such melanopsin variants find use in methods of restoring or enhancing visual function in subject who has experienced photoreceptor loss or retinal detachment due to, e.g., ocular disease, infection, trauma, injury, impact to the head, acute light damage, UV light damage, laser damage, or chemical damage.

### Definitions

The compositions and methods described herein may employ, unless otherwise indicated, conventional techniques and descriptions of molecular biology (including recombinant techniques), cell biology, biochemistry, immunochemistry, and ophthalmic techniques, which are within the skill of those who practice in the art. Such conventional techniques include methods for observing and analyzing the retina, or vision in a subject, cloning and propagation of recombinant virus, formulation of a pharmaceutical composition, and biochemical purification and immunochemistry. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3rd Ed., W.H. Freeman Pub., New York (2000); and Berg et al., Biochemistry, 5th Ed., W.H. Freeman Pub., New York (2002), all of which are herein incorporated by reference in their entirety for all purposes.

Before describing the embodiments herein in detail, it is to be understood that the present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The terms “polypeptide,” “protein,” and “peptide” are used interchangeably herein and may refer to polymers of two or more amino acids.

The terms “treat,” “treating”, “treatment,” “ameliorate” or “ameliorating” and other grammatical equivalents as used herein, refer to alleviating, abating or ameliorating an ocular disease or disorder or symptoms of the ocular disease or disorder, preventing additional symptoms of the ocular disease or disorder, ameliorating or preventing the underlying metabolic causes of symptoms, inhibiting the ocular disease or disorder, e.g., arresting the development of the ocular disease or disorder, relieving the ocular disease or disorder, causing regression of the ocular disease or disorder, or stopping the symptoms of the ocular disease or disorder, and are intended to include prophylaxis. The terms further include achieving a therapeutic benefit and/or a prophylactic benefit. The term “therapeutic benefit” refers to eradication or amelioration of the ocular disease or disorder being treated. Also, a therapeutic benefit is achieved with the eradication or amelioration of one or more of the physiological symptoms associated with the ocular disease or disorder such that an improvement is observed in the patient, notwithstanding that, in some embodiments, the patient is still afflicted with the ocular disease or disorder. For prophylactic benefit, the pharmaceutical compositions are administered to a patient who is experiencing vision loss or who is at risk of vision loss, e.g., due to loss of photoreceptor cells, or to a patient reporting one or more of the physiological symptoms of vision loss, e.g., due to loss of photoreceptor cells. Patients with asynchronous development of vision loss may receive therapeutic benefit from treatment of their eye with more advanced vision loss, and prophylactic benefit from treatment of their eye with less advanced vision loss.

The terms “administer,” “administering”, “administration,” and the like, as used herein, can refer to the methods that are used to enable delivery of therapeutics or pharmaceutical compositions to the desired site of biological action. These methods include intravitreal, subretinal, intraocular, or suprachoroidal injection to the eye Other suitable modes of administration are described elsewhere herein.

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

An “vector” or “viral vector” or “recombinant viral vector” as used herein refers to a viral vector (e.g., an adeno-associated vector or “AAV”) or a recombinant viral vector (e.g., a recombinant AAV or “rAAV”) comprising a polynucleotide sequence not of viral origin (e.g., a polynucleotide heterologous to the virus, such as a nucleic acid sequence that encodes a therapeutic transgene, e.g., a melanopsin variant described herein) for transduction into a target cell or to a target tissue. In the case of rAAV, the heterologous polynucleotide is flanked by at least one, and generally by two, AAV inverted terminal repeat sequences (ITRs). An rAAV vector may either be single-stranded (ssAAV) or self-complementary (scAAV). The term “recombinant viral vector” (e.g., rAAV) encompasses both viral vector particles/virions and viral vector plasmids.

The terms “virus,” “viral particle,” “virion,” “recombinant vector particle,” “recombinant particle,” and “recombinant virion” interchangeably refer to a viral particle comprising at least one viral capsid protein and a polynucleotide vector. If the particle comprises a heterologous polynucleotide (e.g., a polynucleotide other than a wild-type viral genome, such as a transgene (e.g., melanopsin variant) to be delivered to a target cell or target tissue), it is typically referred to as a “recombinant vector particle,” “recombinant vector,” or “recombinant virion.” Thus, production of a recombinant viral particle (e.g., rAAV) necessarily includes production of an recombinant polynucleotide vector, as such a vector contained within a recombinant viral particle.

The term “packaging” as used herein can refer to a series of intracellular events that can result in the assembly and encapsidation of a recombinant AAV particle.

AAV “rep” and “cap” genes refer to polynucleotide sequences encoding replication and encapsidation proteins of adeno-associated virus. AAV rep and cap are referred to herein as AAV “packaging genes.”

The term “polypeptide” can encompass both naturally occurring and non-naturally occurring proteins (e.g., a fusion protein), peptides, fragments, mutants, derivatives, and analogs thereof. A polypeptide may be monomeric, dimeric, trimeric, or polymeric. Further, a polypeptide may comprise a number of different domains each of which has one or more distinct activities. For the avoidance of doubt, a “polypeptide” may be any length greater two amino acids.

As used herein, “polypeptide variant” or simply “variant” refers to a polypeptide whose sequence contains an amino acid modification. In some embodiments, the modification is an insertion, duplication, deletion, rearrangement, or substitution of one or more amino acids compared to the amino acid sequence of a reference protein or polypeptide, such as a native or wild-type protein. A variant may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino and carboxy termini. A variant can have the same or a different biological activity compared to the reference protein, or the unmodified protein.

In some embodiments, a variant can have, for example, at least about any one of 80%, 85%, 90%, 91%, 92%, 93%, 94%, 95%, 96%, 97%, 98%, 99% overall sequence homology to its counterpart reference protein. In some embodiments, a variant can have at least about 90% overall sequence homology to the wild-type protein. In some embodiments, a variant exhibits at least about 95%, at least about 98%, at least about 99%, at least about 99.5%, or at least about 99.9% overall sequence identity.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

The term “expression vector” or “expression construct” or “cassette” or “plasmid” or simply “vector” can include any type of genetic construct, including vectors, containing a nucleic acid or polynucleotide coding for a gene product (e.g., a melanopsin variant described herein) in which part or all of the nucleic acid encoding sequence is capable of being transcribed and is adapted for gene therapy. The transcript can be translated into a protein. In some embodiments, the transcript is partially translated or not translated. In certain aspects, expression includes both transcription of a gene and translation of mRNA into a gene product (e.g., a melanopsin variant). In other aspects, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette,” a large number of which are known and available in the art or can be readily constructed from components that are available in the art. In some embodiments, the term “expression vector” refers to both a genetic construct as well as a viral particle that comprises a genetic construct.

The term “heterologous” can refer to an entity that is genotypically distinct from that of the rest of the entity to which it is being compared. For example, a polynucleotide introduced by genetic engineering techniques into a plasmid or vector derived from a different species can be a heterologous polynucleotide. A promoter removed from its native coding sequence and operatively linked to a coding sequence with which it is not naturally found linked can be a heterologous promoter.

As used herein, the terms “subject,” “individual,” and “patient” are used interchangeably to refer to a vertebrate, for example, a mammal. Mammals include, but are not limited to, murines, simians, humans, non-human primates (e.g., cynomolgus monkeys, African green monkeys, macaques, farm animals, sport animals, and pets.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### Melanopsin Variants

### Truncated Melanopsin Variants

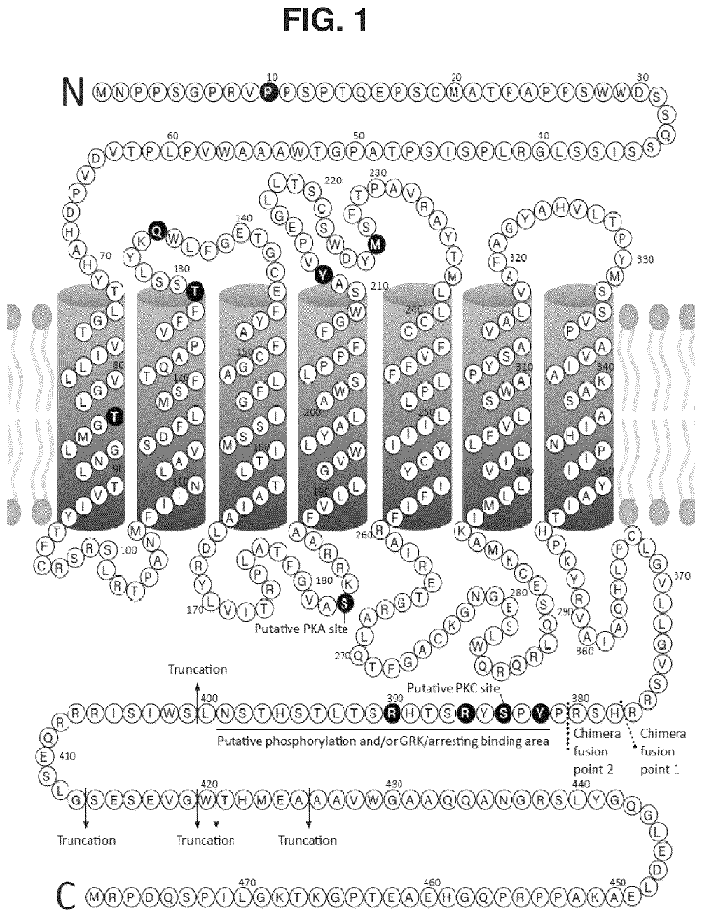

In some embodiments, the present application provides a melanopsin variant comprising no more than amino acids 1-425 of a wild-type human melanopsin set forth in SEQ ID NO: 1, wherein amplitude/conductance (e.g., amplitude/conductance light response) and/or off kinetics (e.g. OFF light response) of the melanopsin variant are greater than the amplitude/conductance and/or faster than the off kinetics of the wild type human melanopsin. Such melanopsin variants are also referred to herein as “truncated melanopsin variants.” FIG. 1 provides a schematic of melanopsin integrated in a cell membrane.

(SEQ ID NO: 1)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVGWTHMEAAAVWGAAQQANGR
SLYGQGLEDLEAKAPPRPQGHEAETPGKTKGLIPSQDPRM
In some embodiments, the melanopsin variant comprises an amino
acid sequence set forth in any one of SEQ ID NOs 2-4.
(SEQ ID NO: 2)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVGW
(SEQ ID NO: 3)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 4)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLG
(SEQ ID NO: 82)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNL
(SEQ ID NO: 83)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISI
(SEQ ID NO: 84)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVGWTHMEA

The sites at which wild-type human melanopsin (SEQ ID NO: 1) was truncated to generate SEQ ID NOs: 2, 3, 4, 82, 83, and 84 are indicated in FIG. 1 with arrows.

In some embodiments, the melanopsin variant is a variant of SEQ ID NO: 2, 3, 4, 82, 83, or 84 that comprises one or more amino acid substitutions relative to the amino acid sequence of SEQ ID NO2, 3, 4, 82, 83, or 84, respectively. In some embodiments, the variant of SEQ ID NO: 2, 3, 4, 82, 83, or 84 comprises one or more amino acid substitution(s) at P10, T83, T129, Q135, S183, Y212, E215, M226, Y382, S384, R386, and/or R390, e.g., in any combination, wherein amino acid position(s) are relative to a reference melanopsin variant set forth in SEQ ID NO: 2, 3, 4, 82, 83, or 84, respectively. In some embodiments, the variant of SEQ ID NO: 2, 3, 4, 82, 83, or 84 comprises at least any one of 2, 3, 4, 5, 6, 7, 8, 9, 10, 11, or 12 substitution(s) at P10, T83, T129, Q135, S183, Y212, E215, M226, Y382, S384, R386, and/or R390, e.g., in any combination, wherein amino acid position(s) are relative to a reference melanopsin variant set forth in SEQ ID NO: 2, 3, 4, 82, 83, or 84, respectively. Each of the amino acid positions that can be substituted is indicated in FIG. 1 by a black circle. In some embodiments, the variant of SEQ ID NO: 2, 3, 4, 82, 83, or 84 comprises one or more substitution(s) selected from the group consisting of: P10F, T83L, T129S, Q135N, S183A, Y212F or Y212A, E215S, M226S or M226T, Y382E or Y382D, S384D, R386A, and R390A or R390D, or any combination thereof. In some embodiments, the variant of SEQ ID NO: 2, 3, 4, 82, 83, or 84 comprises at least any one of 2, 3, 4, 5, 6, 7, 8, 9, 10, 11, or 12 substitution(s) selected from the group consisting of: P10F, T83L, T129S, Q135N, S183A, Y212F or Y212A, E215S, M226S or M226T, Y382E or Y382D, S384D, R386A, and R390A or R390D, or any combination thereof.

In some embodiments, the melanopsin variant comprises a P10F substitution. In some embodiments, the melanopsin variant comprises the sequence set forth in SEQ ID NO: 5. In some embodiments, the melanopsin variant comprises (e.g., further comprises) a T83L substitution. In some embodiments, the melanopsin variant comprises the sequence set forth in SEQ ID NO: 6. In some embodiments, the melanopsin variant comprises (e.g., further comprises) a T129S substitution. In some embodiments, the melanopsin variant comprises the sequence set forth in SEQ ID NO: 7. In some embodiments, the melanopsin variant comprises (e.g., further comprises) a Q135N substitution. In some embodiments, the melanopsin variant comprises the sequence set forth in SEQ ID NO: 8. In some embodiments, the melanopsin variant comprises T129S and Q135N substitutions. In some embodiments, the melanopsin variant comprises the sequence set forth in SEQ ID NO: 9. In some embodiments, the melanopsin variant comprises (e.g., further comprises) a S183A substitution. In some embodiments, the melanopsin variant comprises the sequence set forth in SEQ ID NO: 10. In some embodiments, the melanopsin variant comprises Q135N, and S183A substitutions. In some embodiments, the melanopsin variant comprises the sequence set forth in SEQ ID NO: 11. In some embodiments, the melanopsin variant comprises T129S, Q135N, and S183A substitutions. In some embodiments, the melanopsin variant comprises the sequence set forth in SEQ ID NO: 12. In some embodiments, the melanopsin variant comprises (e.g., further comprises) a Y212F substitution. In some embodiments, the melanopsin variant comprises the sequence set forth in SEQ ID NO: 13. In some embodiments, the melanopsin variant comprises (e.g., further comprises) an M226S or an M226T substitution. In some embodiments, the melanopsin variant comprises the sequence set forth in SEQ ID NO: 14. In some embodiments, the melanopsin variant comprises Y212F and M226S substitutions. In some embodiments, the melanopsin variant comprises the sequence set forth in SEQ ID NO: 15. In some embodiments, the melanopsin variant comprises (e.g., further comprises) a Y382E or a Y382D substitution. In some embodiments, the melanopsin variant comprises the sequence set forth in SEQ ID NO: 16. In some embodiments, the melanopsin variant comprises (e.g., further comprises) an S384D substitution. In some embodiments, the melanopsin variant comprises the sequence set forth in SEQ ID NO: 17. In some embodiments, the melanopsin variant comprises (e.g., further comprises) an R386A substitution. In some embodiments, the melanopsin variant comprises the sequence set forth in SEQ ID NO: 18. In some embodiments, the melanopsin variant comprises S183A, S384D and R386A substitutions. In some embodiments, the melanopsin variant comprises the sequence set forth in SEQ ID NO: 19. In some embodiments, the melanopsin variant comprises (e.g., further comprises) an R390A or an R390D substitution. In some embodiments, the melanopsin variant comprises any one of SEQ ID NO: 20. In some embodiments, the melanopsin variant comprises (e.g., further comprises) Y382D, R386A, and R390A or R390D substitutions. In some embodiments, the melanopsin variant comprises the sequence set forth in SEQ ID NO: 21. In some embodiments, the melanopsin variant comprises (e.g., further comprises) S183A, Y382D, R386A, and R390A or R390D substitutions. In some embodiments, the melanopsin variant comprises the sequence set forth in SEQ ID NO: 22. In some embodiments, the melanopsin variant comprises (e.g., further comprises) T129S, S183A, M226T, Y382D, R386A, and R390A or R390D substitutions. In some embodiments, the melanopsin variant comprises the sequence set forth in SEQ ID NO: 23.

The amino acid sequences of SEQ ID NOs: 5-23 are provided below. Substituted positions in each of SEQ ID NOs: 5-23 are in bold, underlined type.

(SEQ ID NO: 5)
MNPPSGPRVFPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 6)
MNPPSGPRVPPSPTQEPSCMATPAPPSWWDSSQSSISSLGRLPSISPTAPGTWAAAWVPLPTV
DVPDHAHYTLGTVILLVGLLGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 7)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFSSSLYKQWLFGETGCEFYAFCGALFGISSMITLTAIALDRYLVITRPLATFGVASKRRAAFV
LLGVWLYALAWSLPPFFGWSAYVPEGLLTSCSWDYMSFTPAVRAYTMLLCCFVFFLPLLIII
YCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 8)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKNWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 9)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFSSSLYKNWLFGETGCEFYAFCGALFGISSMITLTAIALDRYLVITRPLATFGVASKRRAAFV
LLGVWLYALAWSLPPFFGWSAYVPEGLLTSCSWDYMSFTPAVRAYTMLLCCFVFFLPLLIII
YCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 10)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVAAKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 11)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKNWLFGETGCEFYAFCGALFGISSMITLTAIALDRYLVITRPLATFGVAAKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 12)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFSSSLYKNWLFGETGCEFYAFCGALFGISSMITLTAIALDRYLVITRPLATFGVAAKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 13)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAFVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 14)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYTSFTPAVRAYTMLLCCFVFFLPLLIII
YCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 15)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAFVPEGLLTSCSWDYTSFTPAVRAYTMLLCCFVFFLPLLIII
YCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHRSTLTSHTSNLSWISIRRRQESLGSESEVG
(SEQ ID NO: 16)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPDPSYRSTHRSTLTSHTSNLSWISIRRRQESLGSESEVG
(SEQ ID NO: 17)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPDYRSTHRSTLTSHTSNLSWISIRRRQESLGSESEVG
(SEQ ID NO: 18)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYASTHRSTLTSHTSNLSWISIRRRQESLGSESEVG
(SEQ ID NO: 19)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVAAKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPDYASTHRSTLTSHTSNLSWISIRRRQESLGSESEVG
(SEQ ID NO: 20)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPYPSYRSTHDSTLTSHTSNLSWISIRRRQESLGSESEVG
(SEQ ID NO: 21)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPDPSYASTHDSTLTSHTSNLSWISIRRRQESLGSESEVG
(SEQ ID NO: 22)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVAAKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPDPSYASTHDSTLTSHTSNLSWISIRRRQESLGSESEVG
(SEQ ID NO: 23)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFSSSLYKQWLFGETGCEFYAFCGALFGISSMITLTAIALDRYLVITRPLATFGVAAKRRAAF
VLLGVWLYALAWSLPPFFGWSAYVPEGLLTSCSWDYTSFTPAVRAYTMLLCCFVFFLPLLIII
YCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPDPSYASTHDSTLTSHTSNLSWISIRRRQESLGSESEVG

In some embodiments, the truncated melanopsin variant that comprises one or more substitutions is a substituted truncated variant listed in Table A.

### Chimeric Melanopsin Variants

In some embodiments, the present application provides a melanopsin variant comprising at least amino acids 1-377 of a wild-type human melanopsin set forth in SEQ ID NO: 1 fused (e.g., via peptide bond) to a C-terminal domain (CTD) of a heterologous G protein-coupled receptor (GPCR) or a CTD variant thereof, wherein amplitude/conductance (e.g., amplitude/conductance light response) and/or off kinetics (e.g. OFF light response) of the melanopsin variant are greater than the amplitude/conductance and/or faster than the off kinetics of the wild type human melanopsin. Such melanopsin variants are also referred to herein as “chimeric melanopsin variants.”

In some embodiments, the melanopsin variant comprises amino acids 1-377 of a wild-type human melanopsin set forth in SEQ ID NO: 1. In some embodiments, the melanopsin variant comprises amino acids 1-380 of a wild-type human melanopsin set forth in SEQ ID NO: 1. The chimeric fusion points (i.e., the point at which the N-terminus of a CTD of a GPCR or a CTD variant thereof is attached to the C-terminus at least amino acids 1-377 of melanopsin) are indicated in FIG. 1 with dotted lines.

In some embodiments, the CTD of the heterologous GPCR or CTD variant thereof is the CDT of a visual opsin or a variant thereof. In some embodiments, the CTD of the visual opsin or variant thereof is a CTD of (i) a wild type D. melanogaster rhodopsin 1, (ii) a wild type human rhodopsin, (iii) a wild type human short wavelength opsin (hOPNISW), (iv) a wild type human medium wavelength opsin, or (v) a wild type human long wavelength opsin. In some embodiments, the CTD of the visual opsin comprises an amino acid sequence set forth in any one of SEQ ID NOs: 27-30 and 32-33, shown below. In some embodiments, the CTD of the visual opsin comprises a variant of any one of SEQ ID NOs: 27-30 and 32-33 that comprises one or more amino acid substitutions, deletions, or insertions.

(SEQ ID NO: 27)
TEVSTVSSTQVGPN
(SEQ ID NO: 28)
NKQFQACIMKMVCGKAMTDESDTCSSQKTEVSTVSSTQVGPN
(SEQ ID NO: 29)
AMTDESDTCSSQKTEVSTVSSTQVGPN
(SEQ ID NO: 30)
DAQSQATASEAESKA
(SEQ ID NO: 32)
TEVSSVSSVSPA
(SEQ ID NO: 33)
QFRNCILQLFGKKVDDGSELSSASKTEVSSVSSVSPA

In some embodiments, the N-terminus of the CTD of the heterologous GPCR or CTD variant thereof is fused directly to the C-terminus of the melanopsin variant comprising at least amino acids 1-377 of a wild-type human melanopsin set forth in SEQ ID NO: 1, e.g., via peptide bond. In some embodiments, the melanopsin variant (e.g., chimeric melanopsin variant) comprises an amino acid sequence set forth in any one of SEQ ID NOs: 39-42 and 44-45, shown below. The CTDs of the heterologous GPCRs in each chimeric melanopsin variant are indicated in bold type.

(SEQ ID NO: 39)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRTEVSTVSSTQVGPN
(SEQ ID NO: 40)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRNKQFQACIMKMVCGKAMTDESDTCSSQKTEVSTVSSTQVGPN
(SEQ ID NO: 41)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRAMTDESDTCSSQKTEVSTVSSTQVGPN
(SEQ ID NO: 42)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRPDAQSQATASEAESKA
(SEQ ID NO: 44)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRTEVSSVSSVSPA
(SEQ ID NO: 45)
MNPPSGPRVPPSPTQEPSCMATPAPPSWWDSSQSSISSLGRLPSISPTAPGTWAAAWVPLPTV
DVPDHAHYTLGTVILLVGLTGMLGNLTVIYTFCRSRSLRTPANMFIINLAVSDFLMSFTQAPV
FFTSSLYKQWLFGETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLLCCFVFFLPLLII
IYCYIFIFRAIRETGRALQTFGACKGNGESLWQRQRLQSECKMAKIMLLVILLFVLSWAPYSA
VALVAFAGYAHVLTPYMSSVPAVIAKASAIHNPIIYAITHPKYRVAIAQHLPCLGVLLGVSRR
HSRQFRNCILQLFGKKVDDGSELSSASKTEVSSVSSVSPA

In some embodiments, the chimeric the melanopsin variant comprises an amino acid sequence set forth in any one of SEQ ID NOs: 63, 64, and 65. See Table A.

In some embodiments, the N-terminus of the CTD of the heterologous GPCR or CTD variant thereof is fused to the C-terminus of the melanopsin variant comprising at least amino acids 1-377 of a wild-type human melanopsin set forth in SEQ ID NO: 1 via one or more linker(s). In some embodiments, the one or more linkers comprises one or more peptide linkers, including, but not limited to, e.g., G(4) S (SEQ ID NO: 161), G(4) S) 2 (SEQ ID NO: 162), and G(4) S) 3 (SEQ ID NO: 163), GGGGS (SEQ ID NO: 161), GGGGSGGGGS (SEQ ID NO: 164), or GGGGSGGGGSGGGGS (SEQ ID NO: 165). In some embodiments, the N-terminus of the CTD of the heterologous GPCR or CTD variant thereof is fused to the C-terminus of the melanopsin variant comprising at least amino acids 1-377 of a wild-type human melanopsin set forth in SEQ ID NO: 1 via one or more linker(s) and one or more spacers. See, e.g., Klein et al. (2014) Protein Eng Des Sel. 27 (10): 325-330.

In some embodiments, the melanopsin variant (e.g., a chimeric melanopsin variant described herein) further comprises one or more amino acid substitutions. In some embodiments, the melanopsin variant (e.g., a chimeric melanopsin variant described herein) further comprises one or more substitution(s) at P10, T83, T129, Q135, S183, Y212, E215, M226, Y382, S384, R386, and/or R390, e.g., in any combination, wherein amino acid position(s) are relative to the wild type human melanopsin set forth in SEQ ID NO: 1. In some embodiments, the melanopsin variant (e.g., a chimeric melanopsin variant described herein) further at least any one of 2, 3, 4, 5, 6, 7, 8, 9, 10, 11, or 12 substitution(s) at P10, T83, T129, Q135, S183, Y212, E215, M226, Y382, S384, R386, and/or R390, e.g., in any combination, wherein amino acid position(s) are relative to the wild type human melanopsin set forth in SEQ ID NO: 1. As noted above, each of the amino acid positions that can be substituted is indicated in FIG. 1 by a black circle. In some embodiments, the melanopsin variant (e.g., a chimeric melanopsin variant described herein) further comprises one or more substitution(s) selected from the group consisting of: P10F, T83L, T129S, Q135N, S183A, Y212F or Y212A, E215S, M226S or M226T, Y382E or Y382D, S384D, R386A, and R390A or R390D, or any combination thereof. In some embodiments, the melanopsin variant (e.g., a chimeric melanopsin variant described herein) further comprises at least any one of 2, 3, 4, 5, 6, 7, 8, 9, 10, 11, or 12 substitution(s) selected from the group consisting of: P10F, T83L, T129S, Q135N, S183A, Y212F or Y212A, E215S, M226S or M226T, Y382E or Y382D, S384D, R386A, and R390A or R390D, or any combination thereof. Additionally or alternatively, in some embodiments, the melanopsin variant (e.g., a chimeric melanopsin variant described herein) further comprises one or more amino acid substitutions (e.g., at least any of 1, 2, 3, 4, 5, 6, 7, 8, 9, or 10 amino acid substitutions in the CTD of the heterologous GPCR or variant thereof.

### Substituted Full Length Melanopsin Variants

In some embodiments, the present application provides a melanopsin variant comprising one or more amino acid substitutions at P10, T83, T129, Q135, S183, Y212, E215, M226, Y382, S384, R386, and R390, or any combination thereof, wherein amino acid positions are relative to a wild type human melanopsin set forth in SEQ ID NO: 1, and wherein amplitude/conductance (e.g., amplitude/conductance light response) and/or off kinetics (e.g. OFF light response) of the melanopsin variant are greater than the amplitude/conductance and/or faster than the off kinetics of the wild type human melanopsin. Such melanopsin variants are referred to herein as “substituted full length melanopsin variants.” In some embodiments, the melanopsin variant comprises at least any one of 2, 3, 4, 5, 6, 7, 8, 9, 10, 11, or 12 substitution(s) at P10, T83, T129, Q135, S183, Y212, E215, M226, Y382, S384, R386, and/or R390, e.g., in any combination, wherein amino acid position(s) are relative to the wild type human melanopsin set forth in SEQ ID NO: 1. In some embodiments, the one or more substitutions (e.g., at least any one of 1, 2, 3, 4, 5, 6, 7, 8, 9, 10, 11 or 12 substitutions) are selected from the group consisting of P10F, T83L, T129S, Q135N, S183A, Y212F or Y212A, E215S, M226S or M226T, Y382E or Y382D, S384D, R386A, R390A or R390D. In some embodiments, the melanopsin variant further comprises one or more amino acid substitutions at positions 391-478, wherein amino acid position(s) are relative to the wild type human melanopsin set forth in SEQ ID NO: 1. In some embodiments, the full-length melanopsin variant that comprises one or more substitutions is a substituted variant listed in Table A.

In some embodiments, the melanopsin variant comprises, for example, at least about any one of 80%, 85%, 90%, 91%, 92%, 93%, 94%, 95%, 96%, 97%, 98%, 99% overall sequence homology or identity to any one of SEQ ID NOs: 2-4, 5-23, 39-42, 44-45, 63-65, and 82-84. In some embodiments, the melanopsin variant has at least about 90% overall sequence homology or identity to any one of SEQ ID NOs: 2-4, 5-23, 39-42, 44-45, 63-65, and 82-84. In some embodiments, the melanopsin variant has at least about 95%, at least about 98%, at least about 99%, at least about 99.5%, or at least about 99.9% overall sequence homology or identity to any one of SEQ ID NOs: 2-4, 5-23, 39-42, 44-45, 63-65, and 82-84.

### Functional Characteristics of the Melanopsin Variants

The amplitude/conductance (e.g., amplitude/conductance light response) of the melanopsin variants described herein are greater than the amplitude/conductance of a wild type human melanopsin (e.g., a wild type human melanopsin set forth in SEQ ID NO: 1). In some embodiments the amplitude/conductance of a melanopsin variant described herein is at least about any one of 1.25, 1.5, 1.75, 2, 2.25, 2.5, 2.75, 3, 3.25, 3.5, 3.75, 4, 4.25, 4.5, 4.75, and 5-fold greater than the amplitude/conductance of a wild type human melanopsin. In some embodiments, the amplitude/conductance of a melanopsin variant described herein is at least about any one of 1.25, 1.5, 1.75, 2, 2.25, 2.5, 2.75, 3, 3.25, 3.5, 3.75, 4, 4.25, 4.5, 4.75, and 5-fold greater than the amplitude/conductance of a wild type human melanopsin, as measured in HEK293T cells via calcium release assay, e.g., as described in the Examples herein.

Additionally or alternatively, in some embodiments, the OFF kinetics (e.g., the OFF light response) of the melanopsin variants described herein are faster than the OFF kinetics of a wild type human melanopsin (e.g., a wild type human melanopsin set forth in SEQ ID NO: 1). In some embodiments the OFF kinetics of a melanopsin variant described herein is at least about any one of 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14. 75, or 15-fold faster than the OFF kinetics of a wild type human melanopsin. In some embodiments, the OFF kinetics of a melanopsin variant described herein is at least about any one of 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, or 5-fold faster than the OFF kinetics of a wild type human melanopsin, as measured in HEK293T cells via calcium release assay, e.g., as described in the Examples herein.

### Nucleic Acids, Expression Vectors, Viral Particles, and Host Cells

Also provided herein are nucleic acids that comprise a nucleotide sequence that encodes a melanopsin variant described herein. In some embodiments, nucleotide sequence that encodes a melanopsin variant is operably linked to a transcriptional control element (e.g., a promoter) that provides for expression (e.g., selective expression) of the nucleic acid in a retinal cell (e.g., a retinal ganglion cell, an amacrine cell, a horizontal cell, a bipolar cell, or a photoreceptor cell, such as a rod cell or a cone cell, Müller cells, and retinal pigmented epithelium cells). In some embodiments, the nucleic acid encoding the melanopsin variant is operably linked to a transcriptional control element (e.g., a promoter) that provides for expression in a eukaryotic cell (e.g., a mammalian cell). Exemplary transcriptional control elements and promoters include, but are not limited to ubiquitous or tissue-specific promoters, e.g., a CAG promoter (Miyazaki et al. (1989) Gene 79:269); a cytomegalovirus (CMV) promoter; a human synaptin (hSyn) promoter; a glutamate metabotropic receptor-6 (grm6) promoter (also referred to as a GluR or GluR6 promoter, see Cronin et al. (2014) EMBO Mol. Med. 6:1175); a 4×grm6 promoter (i.e., a four-copy concatemer of a minimal version of the grm6 promoter, see Lagali, et al. (2008) Nat Neurosci 11:667-675 and Masu, et al. (1995) Cell. 80:757-765), an NEFL promoter (Simpson et al. (2019) Human Gene Therapy. 30 (3); 257-272), an SNCG (gamma-synuclein) promoter (Chaffiol et al. (2017) Mol Ther. 25:2546-60), an NEFH promoter (Millington-Ward et al. (2020) Sci Rep. 10:16515), a Pleiades promoter (Portales-Casamar et al. (2010) Proc. Natl. Acad. Sci. USA 107:16589); a choline acetyltransferase (ChAT) promoter (Misawa et al. (1992) J. Biol. Chem. 267:20392); a vesicular glutamate transporter (V-glut) promoter (Zhang et al. (2011) Brain Res. 1377:1); a glutamic acid decarboxylase (GAD) promoter (Rasmussen et al. (2007) Brain Res. 1144:19; Ritter et al. (2016) J. Gene Med. 18:27); a cholecystokinin (CCK) promoter (Ritter et al. (2016) J. Gene Med. 18:27); a parvalbumin (PV) promoter; a somatostatin (SST) promoter; a neuropeptide Y (NPY) promoter; and a vasoactive intestinal peptide (VIP) promoter, a red cone opsin promoter, a rhodopsin promoter, a rhodopsin kinase promoter, a vitelliform macular dystrophy 2 (VMD2) gene promoter, and an interphotoreceptor\retinoid-binding protein (IRBP) gene promoter, an L7 promoter (Oberdick et al. (1990) Science 248:223), a thy-1 promoter, a recoverin promoter (Wiechmann and Howard (2003) Curr. Eye Res. 26:25); a calbindin promoter; and a beta-actin promoter. In some embodiments the promoter is or comprises a synthetic (non-naturally occurring) promoter/enhancer combination. In some embodiments, the promoter comprises the human cytomegalovirus (CMV) immediate-early enhancer and promoter, an adenovirus tripartite leader sequence (TPL) followed by an enhancer element from the major late promoter (eMLP), a synthetic intron, and a Kozak sequence. Such promoter, which is also known as “C11,” is described in Grishanin et al. (2019) Mol Ther 2701): 118-129. Further to the above, in some embodiments, a nucleic acid comprising a sequence that encodes a melanopsin variant described herein comprises (such as further comprises) additional sequences, including, but not limited to, e.g., enhancer(s), intron(s), leader sequence(s), Kozak sequence(s), poly A sequence(s), stuffer sequence(s), woodchuck hepatitis virus post-transcriptional regulatory element (WPRE) sequence(s), and/or one or more inverted terminal repeat (ITR) sequence(s), e.g., AAV ITR sequences, etc.

In some embodiments, a nucleic acid provided herein is in a recombinant genetic construct, e.g., an expression vector. Suitable expression vectors include, but are not limited to, a lentivirus vector, a herpes simplex virus (HSV) vector, an adenovirus vector, a retroviral vector, an adeno-associated virus (AAV) vector, which can be a natural or engineered serotype (e.g., an AAV1, AAV2, AAV3, AAV4, AAV5, AAV6, AAV7, AAV8, AAV9, AAVrh.8, AAVrh.10, AAV11, or AAV12 vector), and the like. In some embodiments, the AAV vector genome is single stranded (ssAAV). In some embodiments, the AAV vector genome is self-complementary (scAAV). In some embodiments, a nucleic acid comprising a nucleotide sequence encoding a melanopsin variant described herein is in a recombinant lentivirus vector, a recombinant HSV, a recombinant adenovirus vector, a recombinant retroviral vector, or a recombinant AAV (“rAAV”) vector (e.g., an rAAV1, rAAV2, rAAV3, rAAV4, rAAV5, rAAV6, rAAV7, rAAV8, rAAV9, rAAVrh.8, rAAVrh.10, rAAV11, or rAAV12 vector).

In some embodiments, the rAAV vector is AAV2.5T.LSV1 (also known as “LSV1”), which is described in detail in WO 2020/180951 and US 2020/0297869, the contents of which are incorporated by reference herein in their entireties. In some embodiments, the rAAV vector is AAV2-4YF, described in Petrs-Silva et al. (2009) Mol. Ther. 17, 463-471 and Petrs-Silva et al. (2011) Mol. Ther. 19, 293-301, the contents of which are incorporated by reference herein in their entireties. In some embodiments, the rAAV vector is AAV2.7m8, described in Dalkara et al. (2013) Sci Transl Med. 5, 189ra76, WO 2012/145601, and US 2014/0364338 the contents of which are incorporated by reference herein in their entireties. In some embodiments, the rAAV vector is AAV9.7m8, described in, e.g., Khabou et al. (2016) Biotechnol Bioeng. (12): 2712-2724 and Khabou et al. (2018) JCI Insight. 3 (2): e96029, the contents of which are incorporated by reference herein in their entireties. In some embodiments, the rAAV vector is R100, described in Kotterman, et al. (2021) “Directed Evolution of AAV Targeting Primate Retina by Intravitreal Injection Identifies R100, a Variant Demonstrating Robust Gene Delivery and Therapeutic Efficacy in Non-Human Primates.” bioRxiv. In some embodiments, the vector comprises a nucleic acid that comprises nucleotide sequence encoding a melanopsin variant described herein and a nucleotide sequence that encodes a variant AAV capsid protein, wherein the variant AAV capsid protein confers infectivity of a retinal cell and/or the ability to cross the inner limiting membrane (ILM) in the eye (e.g., the eye of a mammal, such a mouse, human, or non-human primate). In some embodiments, the AAV capsid protein is AAV.ShH10, AAV.GL, or AAV.NN. Such capsid proteins, and others, are described in detail in, e.g., Day et al. (2014) Adv. Exp. Med. Biol. 801:687; Boye et al. (2016) J. Viral. 90:4215; Vandenberghe and Auricchio (2012) Gene Therapy 19:162; Klimczak et al. (2009) PLOS One 4: e7467; Byrne et al. (2020) “In vivo-directed evolution of adeno-associated virus in the primate retina.” JCI Insight. 2020; 5 (10): e135112; Pavlou et al. (2021) “Novel AAV capsids for intravitreal gene therapy of photoreceptor disorders.” EMBO Mol Med. 13 (4): e13392; Miyadera et al. (2022) “Targeting ON-bipolar cells by AAV gene therapy stably reverses LRIT3-congenital stationary night blindness.” Proc Natl Acad Sci USA. 119 (13): e2117038119; Öztürk et al. (2021) “scAAVengr, a transcriptome-based pipeline for quantitative ranking of engineered AAVs with single-cell resolution.” Elife. 10: e64175, US 2012/0164106; and US 2016/0017295. In some embodiments, a retinal cell is a retinal ganglion cell, an amacrine cell, a horizontal cell, a bipolar cell, or a photoreceptor cell, such as a rod cell or a cone cell, a Müller cell, or a retinal pigmented epithelium cell.

In some embodiments, provided is a virion (i.e., viral particle) comprising (a) a capsid protein and (b) a nucleic acid described herein or expression vector described herein. In some embodiments, the capsid protein is a lentiviral capsid protein, an HSV capsid protein, an adenoviral capsid protein, a retroviral capsid protein, an AAV capsid protein (e.g., an AAV1, AAV2, AAV3, AAV4, AAV5, AAV6, AAV7, AAV8, AAV9, AAVrh.8, AAVrh.10, AAV11, or AAV12 capsid protein), etc. In some embodiments, the capsid protein is a recombinant capsid protein, e.g., a recombinant lentiviral capsid protein, a recombinant HSV capsid protein, a recombinant adenoviral capsid protein, a recombinant retroviral capsid protein, a recombinant AAV capsid protein (e.g., an rAAV1, rAAV2, rAAV3, rAAV4, rAAV5, rAAV6, rAAV7, rAAV8, rAAV9, rAAVrh.8, rAAVrh.10, rAAV11, or rAAV12 capsid protein), etc.

In some embodiments, the virion comprises a variant capsid protein, wherein the variant capsid protein comprises an insertion of a peptide in the capsid protein variable loop 8 relative to a corresponding parental capsid protein, wherein the insertion comprises an amino acid sequence selected from LALGETTRPA (SEQ ID NO: 46); LANETITRPA (SEQ ID NO: 47), LAKAGQANNA (SEQ ID NO: 48), LAKDPKTTNA (SEQ ID NO: 49), KDTDTTR (SEQ ID NO: 50), RAGGSVG (SEQ ID NO: 51), AVDTTKF (SEQ ID NO: 52), STGKVPN (SEQ ID NO: 53), LAKDTDTTRA (SEQ ID NO: 54), LARAGGSVGA (SEQ ID NO: 55), LAAVDTTKFA (SEQ ID NO: 56), and LASTGKVPNA (SEQ ID NO: 57). In some embodiments, the virion comprises a variant capsid protein, wherein the variant capsid protein comprises an insertion of a peptide set forth in any one of SEQ ID NOs: 46-57 at the following amino acid positions to generate the variant capsid: between positions 587 and 588 of the AAV2 capsid protein; between amino acids 590 and 591 of the AAV1 capsid protein; between amino acids 575 and 576 of the AAV5 capsid protein; between amino acids 590 and 591 of the AAV6 capsid protein; between amino acids 589 and 590 of the AAV7 capsid protein; between amino acids 590 and 591 of the AAV8 capsid protein; between amino acids 588 and 589 or 589 and 590 of the AAV9 capsid protein; or between amino acids 589 and 590 of the AAV10 capsid protein. In some embodiments, virion comprises the variant capsid protein AAV2.7m8 (see, e.g., Dalkara et al. (2013) Sci Transl Med. 5, 189ra76, WO 2012/145601, and US 2014/0364338 the contents of which are incorporated by reference herein in their entireties). In some embodiments, virion comprises the variant capsid protein AAV9.7m8 (see Khabou et al. (2016) Biotechnol Bioeng. (12): 2712-2724 and Khabou et al. (2018) JCI Insight. 3 (2): e96029, the contents of which are incorporated by reference herein in their entireties). AAV2.7m8 and AAV9.7m8 are capable of transducing the retina when delivered intravitreally.

In some embodiments, virion comprises a variant capsid protein, wherein the variant capsid protein comprises a modified sequence comprising one or more amino acid substitutions within amino acid residues 570-579 relative to a parental AAV capsid protein, wherein the modified sequence comprises HKFKSGD (SEQ ID NO: 58), and wherein the amino acid residue numbering corresponds to an AAV5 VP1 capsid protein. In some embodiments, the parental AAV capsid protein is an AAV5 capsid protein or an AAV5 and AAV2 hybrid capsid protein. In some embodiments, the parental AAV capsid protein is an AAV2.5T capsid protein. In some embodiments, “AAV2.5T capsid protein” or “AAV2.5T variant” refers to a hybrid capsid protein containing regions from AAV2 and AAV5, described in U.S. Pat. No. 9,441,244, the disclosure of which is incorporated in its entirety. AAV2.5T is capable of transducing the retina when delivered subretinally, but not when injected intravitreally. AAV2.5T transduction may be blocked by the inner limiting membrane (ILM), which is enriched with heparin sulfate proteoglycan (HSPG). The surface-exposed domains of AAV2.5T are identical to that of AAV5 except for a single substitution of A to T in aa582 of AAV2.5T (aa581 of AAV5), a mutation which appears to increase infectivity in mammalian cells without impacting AAV5's typical sialic acid receptor binding. AAV5 and AAV2.5T have negligible heparin sulfate binding, whereas AAV2 has high affinity for heparin sulfate. In some embodiments, the parental AAV capsid protein is an AAV2.5T VP1 capsid protein. In some embodiments, the modified sequence comprises LAHKFKSGDA (SEQ ID NO: 59). In some embodiments, the rAAV is AAV2.5T.LSV1. In some embodiments, “AAV2.5T.LSV1” or “AAV2.5T.LSV1 variant” refers to a rAAV variant that comprises a variant capsid protein, wherein the variant capsid protein comprises a loop substitution variant, wherein the loop substitution variant comprises the amino acid loop sequence LAHKFKSGDA (SEQ ID NO:60) at amino acid residues 570-579 relative to AAV2.5T, the parental AAV capsid protein. In some embodiments, the virion comprises an AAV2-4YF capsid protein (see Petrs-Silva et al. (2009) Mol. Ther. 17, 463-471 and Petrs-Silva et al. (2011) Mol. Ther. 19, 293-301, the contents of which are incorporated by reference herein in their entireties), or an R100 capsid protein (see Kotterman, et al. (2021) “Directed Evolution of AAV Targeting Primate Retina by Intravitreal Injection Identifies R100, a Variant Demonstrating Robust Gene Delivery and Therapeutic Efficacy in Non-Human Primates.” BioRxiv, the contents of which are incorporated herein by reference in their entirety).

In some embodiments, the virion comprises a variant capsid protein, wherein the variant capsid protein comprises an insertion of a peptide in the capsid protein variable loop 8 relative to a corresponding parental capsid protein, wherein the insertion comprises an amino acid sequence selected from LAHQDTTKNS (SEQ ID NO: 85), LALGETTRAA (SEQ ID NO: 86), LAHQDTTRPA (SEQ ID NO: 87), LARQDTTKNA (SEQ ID NO: 88), LAHQDSTKNA (SEQ ID NO: 89), LAHQDATKNA (SEQ ID NO: 90), LAHQDTTKPA (SEQ ID NO: 91), IALSETTRPA (SEQ ID NO: 92), LAHQDTTKKC (SEQ ID NO: 93), LALGEATRPA (SEQ ID NO: 94), LALGETTRTA (SEQ ID NO: 95), LALSEATRPA (SEQ ID NO: 96), LAKDETKNSA (SEQ ID NO: 97), LALGETTKPA (SEQ ID NO: 98), LAHQATTKNA (SEQ ID NO: 99), LQRGNRQTTTADVNTQ (SEQ ID NO: 100), LQRGNRQATTADVNTL (SEQ ID NO: 101), LQRGNRQATTEDVNTQ (SEQ ID NO: 102), LQRGNRQAATEDVNTQ (SEQ ID NO: 103), LQRGNRQAATADVNSL (SEQ ID NO: 104), LQRGNRQAATADVNKL (SEQ ID NO: 105), LQRGVRVPSVLEVNGQ (SEQ ID NO: 106), LQRGNRQAATADVNIL (SEQ ID NO: 107), LQRGKRQATTADVNTQ (SEQ ID NO: 108), LHRGNRQAATADVNTL (SEQ ID NO: 109), SRTNTPSGTTTQPTLQFSQ (SEQ ID NO: 110), SKTDTPSGTTTQSRLQFSQ (SEQ ID NO: 111), SRTDTPSETTTQSRLQFSQ (SEQ ID NO: 112), SRTNSPSGTTTQSSLQFSQ (SEQ ID NO: 113), SRTDIPSGTTTQSRLQFSQ (SEQ ID NO: 114), HQDTTKN (SEQ ID NO: 115), LGETTRA (SEQ ID NO: 116), HQDTTRP (SEQ ID NO: 117), RQDTTKN (SEQ ID NO: 118), HQDSTKN (SEQ ID NO: 119), HQDATKN (SEQ ID NO: 120), HQDTTKP (SEQ ID NO: 121), LSETTRP (SEQ ID NO: 122), HQDTTKK (SEQ ID NO: 123), LGEATRP (SEQ ID NO: 124), LGETTRT (SEQ ID NO: 125), LSEATRP (SEQ ID NO: 126), KDETKNS (SEQ ID NO: 127), LGETTKP (SEQ ID NO: 128), and HQATTKN (SEQ ID NO: 129). For example, in some embodiments, the insertion site is between amino acids 587 and 588 of AAV2, between amino acids 590 and 591 of AAV1, between amino acids 575 and 576 of AAV5, between amino acids 590 and 591 of AAV6, between amino acids 589 and 590 ofAAV7, between amino acids 590 and 591 of AAV8, between amino acids 588 and 589 of AAV9, between amino acids 588 and 589 of AAV10, or between amino acids 585 and 586 of AAV4. Additional details regarding such variant capsid proteins, and viral particles (e.g., recombinant viral particles) comprising such variant capsid proteins, are provided in WO 2021/243085 and US 2021/0371879, the contents of which are incorporated by reference herein in their entireties.

In some embodiments, the virion comprises a variant capsid protein, wherein the variant capsid protein comprises a peptide insertion in the GH-loop relative to a corresponding parental capsid protein, wherein the insertion is selected from the group consisting of ISDQTKH (SEQ ID NO: 130), QADTTKN (SEQ ID NO: 131), ASDSTKA {SEQ ID NO: 132), NQDYTKT (SEQ ID NO: 133), HDITKNI (SEQ ID NO: 134), HPDTTKN (SEQ TD NO: 135), HQDTTKN (SEQ ID NO: 136), NKITNKD (SEQ ID NO: 137), ISNENEH (SEQ ID NO: 138), QANANEN (SEQ ID NO: 139), GKSKVID (SEQ ID NO: 140), TNRTSPD (SEQ ID NO: 141), PNSTHGS (SEQ ID NO: 142), KDRAPST (SEQ ID NO: 143), LAQADITKNA (SEQ ID NO: 144), LAISDQTKHA (SEQ ID NO: 145), LGISDQTKHA (SEQ ID NO: 146), LAASDSTKAA (SEQ ID NO: 147), LANQDYTKTA (SEQ ID NO: 148), LAHDITKNIA (SEQ ID NO: 149), LAHPDTTKNA (SEQ ID NO: 150), LAHQDTTKNA (SEQ ID NO: 151), LANKTTNKDA (SEQ ID NO: 152), LPISNENEHA (SEQ ID NO: 153), LPQANANENA (SEQ ID NO: 154), LAGKSKVIDA (SEQ ID NO: 155), LATNRTSPDA (SEQ ID NO: 156), LAPNSTHGSA (SEQ ID NO: 157) and LAKDRAPSTA (SEQ ID NO: 158). For example, in some embodiments, the insertion site is between amino acids 587 and 588 of AAV2, between amino acids 590 and 591 of AAV1, between amino acids 575 and 576 of AAV5, between amino acids 590 and 591 of AAV6, between amino acids 589 and 590 ofAAV7, between amino acids 590 and 591 of AAV8, between amino acids 588 and 589 of AAV9, between amino acids 588 and 589 of AAV10, or between amino acids 585 and 586 of AAV4. Additional details regarding such variant capsid proteins, and viral particles (e.g., recombinant viral particles) comprising such variant capsid proteins, are provided in WO 2017/19735S, the contents of which are incorporated by reference herein in their entireties.

Also provided herein are host cells for producing virions described herein. Host cells comprise a nucleic acid provided herein. Suitable host cells, including, for example, human-derived cell lines such as HeLa, A549, or 293 cells, or insect-derived cell lines such as SF-9. In some embodiments, the host cell further comprises a polynucleotide encoding a capsid protein (e.g., any of the capsid proteins described herein), a polynucleotide encoding a rep protein, and AAV helper functions. In some embodiments, a nucleic acid described herein further comprises the polynucleotide encoding the capsid protein and the polynucleotide encoding the rep protein. In some embodiments, a nucleic acid described herein does not comprise the polynucleotide encoding the capsid protein and the polynucleotide encoding the rep protein.

### Production of Recombinant Viral Particles

Numerous methods are known in the art for production of rAAV vectors, including transfection, stable cell line production, and infectious hybrid virus production systems which include adenovirus-AAV hybrids, herpesvirus-AAV hybrids (Conway, et al. (1997) J. Virology 71 (11): 8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as Sf9, e.g., rhabdovirus-free Sf9 cells, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a therapeutic transgene) flanked by at least one AAV ITR sequence (more typically two ITR sequences); and 5) suitable media and media components to support rAAV production. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

Suitable rAAV production culture media of the present invention may be supplemented with serum or serum-derived recombinant proteins at a level of 0.5%-20% (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

In some embodiments, the rAAV production culture harvest is clarified to remove host cell debris. In some embodiments, the production culture harvest is clarified by filtration through a series of depth filters including, for example, a grade DOHC Millipore Millistak+ HC Pod Filter, a grade A1HC Millipore Millistak+ HC Pod Filter, and a 0.2 μm Filter Opticap XL Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art. In some embodiments, the rAAV production culture harvest is further treated to digest any high molecular weight DNA present in the production culture.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below to purify rAAV particles are found, for example, in Xiao et al . . . (1998) Journal of Virology 72:2224-2232; U.S. Pat. Nos. 6,989,264 and 8,137,948 and WO 2010/148143. Methods to purify adenovirus particles are found, for example, in Bo, H et al., (2014) Eur. J. Pharm. Sci. 67C: 119-125. Methods to purify lentivirus particles are found, for example, in Segura et al . . . (2013) Expert Opin Biol Ther. 13 (7): 987-1011. Methods to purify HSY particles are found, for example, in Goins, W F et al., (2014) Herpes Simplex Virus Methods in Molecular Biology 1144:63-79.

### Pharmaceutical Compositions and Kits

In some embodiments, provided is a pharmaceutical composition comprising: a) a nucleic acid comprising a nucleotide sequence that encodes a melanopsin variant herein, an expression vector described herein, or a virion comprising a nucleic acid or expression vector described herein, and b) a pharmaceutically acceptable excipient or carrier. In some embodiments “pharmacologically acceptable excipient or carrier” refers to any excipient, carrier, diluent, stabilizer, etc., that has substantially no long-term or permanent detrimental effect when administered to a subject (e.g., a mammal, such as a mouse, a human, or a non-human primate). Typically, such excipient is mixed with an active compound (e.g., a nucleic acid disclosed herein, an expression vector disclosed herein, or a viral particle disclosed herein), or permitted to dilute or enclose the active compound and can be a solid, semi-solid, or liquid agent. It is understood that the active ingredients can be soluble or can be delivered as a suspension in the desired excipient or diluent. Any of a variety of pharmaceutically acceptable excipients can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable excipient can depend on the mode of administration. Except insofar as any pharmacologically acceptable excipient is incompatible with the active ingredient, its use in pharmaceutically acceptable compositions is contemplated. Some examples of materials that can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and other waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations. Other non-limiting examples of specific uses of such pharmaceutical carriers can be found in “Pharmaceutical Dosage Forms and Drug Delivery Systems” (Howard C. Ansel et al., eds., Lippincott Williams & Wilkins Publishers, 7th ed. 1999); “Remington: The Science and Practice of Pharmacy” (Alfonso R. Gennaro ed., Lippincott, Williams & Wilkins, 20th 2000); “Goodman & Gilman's The Pharmacological Basis of Therapeutics 13th ed.” Brunton et al., eds., McGraw-Hill Professional, 2017); and “Handbook of Pharmaceutical Excipients” (Sheskey et al., APhA Publications, 9th edition 2020).

In some embodiments, the pharmaceutical composition further comprises one or more additional pharmaceutically acceptable component(s), e.g., buffers, preservatives, tonicity adjusters, salts, antioxidants, physiological substances, pharmacological substances, bulking agents, emulsifying agents, wetting agents, and the like. Various buffers and means for adjusting pH can be used to prepare a pharmaceutical composition, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a pharmaceutical composition.

In some embodiments, a nucleic acid, expression vector, or virion described herein is formulated with one or more biocompatible polymers. In some embodiments, a nucleic acid, expression vector, or virion described herein is formulated in a liposome. See, e.g., US 2017/0119666. In some embodiments, a nucleic acid, expression vector, or virion described herein is formulated in a nanoparticle. Nanoparticles include, e.g., polyalkylcyanoacrylate nanoparticles, nanoparticles comprising poly(lactic acid), nanoparticles comprising poly(lactic-co-glycolic acid) (PLGA) nanoparticles, and the like. In some embodiments, a nucleic acid, expression vector, or virion described herein is formulated in a hydrogel. Suitable hydrogel components include, but are not limited to, silk (see, e.g., U.S. Patent Publication No. 2017/0173161), poly(lactic acid) (PLA), poly(glycolic acid) (PGA), poly(lactide-co-glycolide) (PLGA), polyesters, hyaluronic acid, and the like. In some embodiments, a nucleic acid, expression vector, or virion described herein is present in a buffered saline solution. In some embodiments, the buffered saline solution between about 50 μL to 1000 μL in volume, including any range in between these values. In some embodiments, the 50 μL to 1000 μL in volume contains a unit dose of a nucleic acid, expression vector, or virion described herein.

### Methods of Restoring or Enhancing Visual Function

In some embodiments, provided is a method of restoring or enhancing visual function in a subject, comprising administering a nucleic acid described herein, an expression vector described herein, a virion described herein, or a pharmaceutical composition described herein to the eye of a subject. In some embodiments, the subject is a mammal, e.g., a mouse, a human, or a non-human primate (e.g., a macaque or cynomolgus monkey). In some embodiments, the nucleic acid, expression vector, virion, or pharmaceutical composition described herein is administered via intravitreal (IVT) injection, subretinal (SR) injection, intraocular injection, or suprachoroidal injection. Other suitable modes of administration include, e.g., periocular injection, subconjunctive injection, retrobulbar injection, injection into the sclera, and intercameral injection. In some embodiments, the nucleic acid, expression vector, virion, or pharmaceutical composition described herein is administered to the subject over a period of time ranging between about 1 day to about 1 year, including any range between these values (e.g., between about one week to about two weeks, between about two weeks and about 1 month, between about one month to and about 6 months, between about 6 months to about 1 year). In some embodiments, the nucleic acid, expression vector, virion, or pharmaceutical composition described herein is administered to the subject over a period of time that is greater than one year. Following administration of the nucleic acid, expression vector, virion, or pharmaceutical composition, a melanopsin variant described herein is produced in the retinal cell (e.g., a retinal ganglion cell, an amacrine cell, a horizontal cell, a bipolar cell, or a photoreceptor cell, such as a rod cell or a cone cell, Müller cell, or retinal pigmented epithelium cell), and expression of the melanopsin variant in the retinal cell provides for enhanced or restored visual function in the subject. Tests for visual function are known in the art, and any known test can be applied to assess visual function in a subject administered with a nucleic acid, expression vector, virion, or pharmaceutical composition described herein.

In some embodiments, the subject is a human subject. In some embodiments, the human subject has a degenerative disease or a disorder that affects the retina. In some embodiments, the human subject has reduced sensitivity to light. In some embodiments, the neurons in the subject's retinal circuit (e.g., bipolar cells, amacrine interneurons and/or ganglion cells that output to the brain) are made directly sensitive to light by expression of a melanopsin variant described herein in the subject's retinal cells. In some embodiments, the human subject has reduced visual function due to loss of rod and cone photoreceptors. In some embodiments, the subject has an inherited retinal degenerative disease (IRD), such as Leber's Congenital Amaurosis (LCA), retinitis pigmentosa, Usher syndrome, Stargardt disease, cone-rod dystrophy (CRD), achromatopsia, choriodermia, retinoschisis, or Bardet-Beidl syndrome. In some embodiments, the human subject has an ocular disease. In some embodiments, the human subject has an ocular disease, including, but not limited to, e.g., macular degeneration (such as age-related macular degeneration), diabetic retinopathy, geographic atrophy, and cone dystrophy. In some embodiments the human individual has retinal damage or retinal detachment due to injury (e.g., blunt trauma, blast injury, impact to the head, acute light damage, UV light damage, laser damage, chemical damage, etc.). In some embodiments the human individual has retinal damage or retinal detachment due to infection.

### Kits and Articles of Manufacture

In some embodiments, provided are kits or articles of manufacture that comprise one or more nucleic acids, expression vectors, virions, or pharmaceutical compositions disclosed herein for use according to a method or restoring or enhancing visual function described herein. In some embodiments, the kit comprises a lyophilized form of a pharmaceutical composition and a solution for reconstituting the pharmaceutical composition prior to administration to a subject. In some embodiments, the kit further comprises instructions for administering the one or more nucleic acids, expression vectors, virions, or pharmaceutical compositions herein to the eye of a subject (e.g., a human subject) via intravitreal injection, subretinal injection, intraocular injection, suprachoroidal injection, or other route of administration described herein.

In some embodiments, the kit comprises pharmaceutically acceptable excipients, buffers, solutions, etc. for administering the pharmaceutical composition. In some embodiments, the kit further comprises instructions for suitable operational parameters in the form of a label or a separate insert. For example, the kit may have standard instructions informing a physician or laboratory technician to prepare a therapeutically effective dose of the nucleic acid, expression vector, virion, or pharmaceutical composition and/or to reconstitute lyophilized compositions. In some embodiments, the kit further comprises a device for administration, such as a syringe, filter needle, extension tubing, cannula, or other implements to facilitate injection of the pharmaceutical composition to the eye of a subject. Exemplary injection routes are described elsewhere herein. In some embodiments, the kit comprises a pharmaceutical composition in the form of a suspension or refrigerated suspension, and a syringe and/or a buffer for dilution. In some embodiments, the kit comprises a pre-filled syringe comprising the suspension or refrigerated suspension.

All publications and patent applications cited in this specification are herein incorporated by reference as if each individual publication or patent application were specifically and individually indicated to be incorporated by reference.

The preceding description is presented to enable a person of ordinary skill in the art to make and use the various embodiments. Descriptions of specific compositions, techniques, and applications are provided only as examples. Various modifications to the embodiments described above will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

### EXAMPLES

The present disclosure will be more fully understood by reference to the following examples. The examples should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1A: Characterization of the Amplitude/Conductance Light Responses and the OFF Light Responses of Melanopsin Variants

TABLE A

| | Description | SEQ |
|---|---|---|
| Name | (N-terminus to C-terminus) | ID NO. |
| Opn4/WT | Wild type (WT) human melanopsin | 1 |
| 396aa | Truncated melanopsin variant, amino acids 1-396 of WT human | |
| | melanopsin | |
| 413aa | Truncated melanopsin variant, amino acids 1-413 of WT human | 4 |
| | melanopsin | |
| 419aa | Truncated melanopsin variant, amino acids 1-419 of WT human | 3 |
| | melanopsin | |
| 401aa-7aa | Truncated melanopsin variant, amino acids 1-400 of WT human | |
| | melanopsin, amino acids 393-390 removed | |
| 380aa | Truncated melanopsin variant, amino acids 1-380 of WT human | |
| | melanopsin | |
| 389aa | Truncated melanopsin variant, amino acids 1-389 of WT human | |
| | melanopsin | |
| 400aa | Truncated melanopsin variant, amino acids 1-400 of WT human | 82 |
| (Also referred to | melanopsin | |
| as “L400STOP”) | | |
| 405aa | Truncated melanopsin variant, amino acids 1-405 of WT human | 83 |
| (Also referred to | melanopsin | |
| as “I405STOP”) | | |
| 420aa | Truncated melanopsin variant, amino acids 1-420 of WT human | 2 |
| | melanopsin | |
| 425aa | Truncated melanopsin variant, amino acids 1-425 of WT human | 84 |
| (Also referred to | melanopsin | |
| as “A425STOP”) | | |
| MW | Amino acids 1-353 of WT human melanopsin (plain text) + 39 | 59 |
| | amino acids of the CTD of WT human medium wavelength opsin | |
| | (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITNRQFRNC | |
| | ILQLFGKKVD DGSELSSASK TEVSSVSSVS PA | |
| WT-MW1 | Amino acids 1-367 of WT human melanopsin (plain text) + 32 | 60 |
| | amino acids of the CTD of WT human medium wavelength opsin | |
| | (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCILQ LFGKKVDDGS ELSSASKTEV SSVSSVSPA | |
| WT-MW2 | Amino acids 1-375 of WT human melanopsin (plain text) + 26 | 61 |
| | amino acids of the CTD of WT human medium wavelength opsin | |
| | (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSKKVDD GSELSSASKT EVSSVSSVS | |
| | PA | |
| WT-MW3 | Amino acids 1-377 of WT human melanopsin (plain text) + 24 | 62 |
| | amino acids of the CTD of WT human medium wavelength opsin | |
| | (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRVDD GSELSSASKT EVSSVSSVSP | |
| | A | |
| WT-MW4 | Amino acids 1-380 of WT human melanopsin + SEQ ID NO: 32 | 44 |
| | (from CTD of WT human medium wavelength opsin) | |
| | WVPLPTVDVP DHAHYTLGTV ILLVGLTGML GNLTVIYTFC | |
| | RSRSLRTPAN MFIINLAVSD FLMSFTQAPV FFTSSLYKQW | |
| | LFGETGCEFY AFCGALFGIS SMITLTAIAL DRYLVITRPL | |
| | ATFGVASKRR AAFVLLGVWL YALAWSLPPF FGWSAYVPEG | |
| | LLTSCSWDYM SFTPAVRAYT MLLCCFVFFL PLLIIIYCYI | |
| | FIFRAIRETG RALQTFGACK GNGESLWQRQ RLQSECKMAK | |
| | IMLLVILLFV LSWAPYSAVA LVAFAGYAHV LTPYMSSVPA | |
| | VIAKASAIHN PIIYAITHPK YRVAIAQHLP CLGVLLGVSR | |
| | RHSRTEVSSV SSVSPA | |
| Rho | Amino acids 1-353 of WT human melanopsin (plain text) + 39 | 63 |
| | amino acids of the CTD of WT human rhodopsin (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITNKQFRNC | |
| | MLTTICCGKN PLGDDEASAT VSKTETSQVA PA | |
| DMrho1 | Amino acids 1-353 of WT human melanopsin (plain text) + 41 | 64 |
| | amino acids of the CTD of D. melanogaster rhodopsin (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRLA | |
| | LKEKCPCCVF GKVDDGKSSD AQSQATASEA ESKA | |
| WT_DMrhol | Amino acids 1-368 of WT human melanopsin (plain text) + 26 | 65 |
| | amino acids of the CTD of D. melanogaster rhodopsin (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLVF GKVDDGKSSD AQSQATASEA ESKA | |
| DMrho1-MW1 | Amino acids 1-353 of WT human melanopsin (plain text) + 13 | 66 |
| | amino acids of the CTD of D. melanogaster rhodopsin (bold text) + | |
| | 33 amino acids of the CTD of WT human medium wavelength opsin | |
| | (underlined text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRLA | |
| | LKEKCPCILQ LFGKKVDDGS ELSSASKTEV SSVSSVSPA | |
| DMrho1-MW2 | Amino acids 1-353 of WT human melanopsin + 20 amino acids of | 67 |
| | the CTD of D. melanogaster rhodopsin (bold text) + 26 amino acids | |
| | of the CTD of WT human medium wavelength opsin (underlined | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRLA | |
| | LKEKCPCCVF GKVKKVDDGS ELSSASKTEV SSVSSVSPA | |
| DMrho1-MW3 | Amino acids 1-353 of WT human melanopsin + 26 amino acids of | 68 |
| | the CTD of D. melanogaster rhodopsin (bold text) + 19 amino acids | |
| | of the CTD of WT human medium wavelength opsin (underlined | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQMNPPSGP RVPPSPTQEP SCMATPAPPS WWDSSQSSIS | |
| | SLGRLPSISP TAPGTWAAAW VPLPTVDVPD HAHAPVFFTS | |
| | SLYKQWLEGE TGCEFYAFCG ALFGISSMIT LTAIALDRYL | |
| | VITRPLATFG VASKRRAAFV LLGVWLYALA WSLPPFFGWS | |
| | AYVPEGLLTS CSWDYMSFTP AVRAYTMLLC CFVFFLPLLI | |
| | IIYCYIFIFR AIRETGRALQ TFGACKGNGE SLWQRQRLQS | |
| | ECKMAKIMLL VILLFVLSWA PYSAVALVAF AGYAHVLTPY | |
| | MSSVPAVIAK ASAIHNPIIY AITHPKYRLA LKEKCPCCVF | |
| | GKVDDGKSSE LSSASKTEVS SVSSVSPA | |
| SW | Amino acids 1-353 of WT human melanopsin (plain text) + 42 | 69 |
| | amino acids of the CTD of WT human short wavelength opsin (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITNKQFQAC | |
| | IMKMVCGKAM TDESDTCSSQ KTEVSTVSST QVGPN | |
| WT-SW1 | Amino acids 1-367 of WT human melanopsin (plain text) + 35 | 70 |
| | amino acids of the CTD of WT human short wavelength opsin (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIER AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCIMK MVCGKAMTDE SDTCSSQKTE VSTVSSTQVG | |
| | PN | |
| WT-SW2 | Amino acids 1-375 of WT human melanopsin (plain text) + 28 | 71 |
| | amino acids of the CTD of WT human short wavelength opsin (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSKAMTD ESDTCSSQKT EVSTVSSTQV | |
| | GPN | |
| WT-SW3 | Amino acids 1-377 of WT human melanopsin (plain text) + 26 | 72 |
| | amino acids of the CTD of WT human short wavelength opsin (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRMTD ESDTCSSQKT EVSTVSSTQV | |
| | GPN | |
| WT-SW4 | Amino acids 1-380 of WT human melanopsin + SEQ ID NO: 27 | 39 |
| | (from CTD of WT human short wavelength opsin) | |
| WT2_DMrhol | Amino acids 1-381 of WT human melanopsin + SEQ ID NO: 30 | 42 |
| | (from CTD of WT D. melanogaster rhodopsin) | |
| 380A1R-1 | Amino acids 1-380 of WT human melanopsin (plain text) + 34 | 43 |
| | amino acids from CTD of WT human adenosine receptor A1 (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRHSR QKFRVTFLKI WNDHFRCQPA | |
| | PPIDEDLPEE RPDD | |
| 380A1R-2 | Amino acids 1-380 of WT human melanopsin (plain text) + 30 | 73 |
| | amino acids from CTD of WT human adenosine receptor A1 (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRHSR VTFLKIWNDH FRCQPAPPID | |
| | EDLPEERPDD | |
| 380mGluR5-1 | Amino acids 1-380 of WT human melanopsin (plain text) + 40 | 38 |
| | amino acids from CTD of WT human metabotropic glutamate | |
| | receptor 5 (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRHSR ERNVRSAFTT STVVRMHVGD | |
| | GKSSSAASRS SSLVNLWKRR | |
| 380OPN1MW-1 | Amino acids 1-380 of WT human melanopsin + SEQ ID NO: 33 | 45 |
| | (from CTD of WT human medium wavelength opsin) | |
| 380OPN1MW-2 | Amino acids 1-380 of WT human melanopsin (plain text) + 24 | 74 |
| | amino acids from CTD of WT human medium wavelength opsin | |
| | (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRHSR VDDGSELSSAS KTEVSSVSSV | |
| | SPA | |
| 380OPNISW-1 | Amino acids 1-380 of WT human melanopsin + SEQ ID NO: 28 | 40 |
| | (from WT human short wavelength opsin) | |
| 380OPN1SW-2 | Amino acids 1-380 of WT human melanopsin + SEQ ID NO: 29 | 41 |
| | (from WT human short wavelength opsin) | |
| 380V2R-1 | Amino acids 1-380 of WT human melanopsin + SEQ ID NO: 24 | 35 |
| | (from CTD of WT human vasopressin 2 receptor) | |
| 380V2R-2 | Amino acids 1-380 of WT human melanopsin + SEQ ID NO: 25 | 36 |
| | (from CTD of WT human vasopressin 2 receptor) | |
| 3795HT2a | Amino acids 1-379 of WT human melanopsin (plain text) + 87 | 75 |
| | amino acids from WT human h5-HT_{2A} CTD (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRHSK TYRSAFSRYI QCQYKENKKP | |
| | LQLILVNTIP ALAYKSSQLQ MGQKKNSKQD AKTTDNDCSM | |
| | VALGKQHSEE ASKDNSDGVN EKVSCV | |
| V1mwOpsinCTD | Amino acids 1-355 of WT human melanopsin (plain text) + 37 | 76 |
| | amino acids from WT human medium wavelength opsin CTD (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPQFRNC | |
| | ILQLFGKKVD DGSELSSASK TEVSSVSSVS PA | |
| V2mwOpsinCTD | Amino acids 1-377 of WT human melanopsin (plain text) + 37 | 77 |
| | amino acids from WT human medium wavelength opsin CTD (bold | |
| | text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRQFR NCILQLFGKK VDDGSELSS | |
| | ASKTEVSSVS SVSPA | |
| V1mwGluR5CTD | Amino acids 1-355 of WT human melanopsin (plain text) + 40 | 159 |
| | amino acids from CTD of WT human metabotropic glutamate | |
| | receptor 5 (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPERNVR | |
| | SAFTTSTVVR MHVGDGKSSS AASRSSSLVN LWKRR | |
| V2mwGluR5CTD | Amino acids 1-377 of WT human melanopsin (plain text) + 40 | 37 |
| | amino acids from CTD of WT human metabotropic glutamate | |
| | receptor 5 (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRERN VRSAFTTSTV VRMHVGDGKS | |
| | SSAASRSSSL VNLWKRR | |
| V1V2RCTD | Amino acids 1-355 of WT human melanopsin (plain type) + 29 | 160 |
| | amino acids from CTD of human vasopressin 2 receptor (bold type) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPARGRT | |
| | PPSLGPQDES CTTASSSLAK DTSS | |
| V2V2RCTD | Amino acids 1-377 of WT human melanopsin (plain type) + 29 | 34 |
| | amino acids from CTD of human vasopressin 2 receptor (bold type) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRARG RTPPSLGPQD ESCTTASSSL | |
| | AKDTSS | |
| V15HT2ARCTD | Amino acids 1-355 of WT human melanopsin (plain text) + 87 | 78 |
| | amino acids from WT human h5-HT_{2A} CTD (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKTYRS | |
| | AFSRYIQCQY KENKKPLQLI LVNTIPALAY KSSQLQMGQK | |
| | KNSKQDAKTT DNDCSMVALG KQHSEEASKD NSDGVNEKVS | |
| | CV | |
| V25HT2ARCTD | Amino acids 1-377 of WT human melanopsin (plain text) + 34 | 79 |
| | amino acids from WT human AIR CTD (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLFGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRKTY RSAFSRYIQC QYKENKKPLQ | |
| | LILVNTIPAL AYKSSQLQMG QKKNSKQDAK TTDNDCSMVA | |
| | LGKQHSEEAS KDNSDGVNEK VSCV | |
| VIAIR | Amino acids 1-355 of WT human melanopsin (plain text) + 87 | 80 |
| | amino acids from WT human AIR CTD (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDELMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPQKFRV | |
| | TFLKIWNDHF RCQPAPPIDE DLPEERPDD | |
| V2A1R | Amino acids 1-377 of WT human melanopsin (plain text) + 87 | 81 |
| | amino acids from WT human h5-HT_{2A} CTD (bold text) | |
| | MNPPSGPRVP PSPTQEPSCM ATPAPPSWWD SSQSSISSLG | |
| | RLPSISPTAP GTWAAAWVPL PTVDVPDHAH YTLGTVILLV | |
| | GLTGMLGNLT VIYTFCRSRS LRTPANMFII NLAVSDFLMS | |
| | FTQAPVFFTS SLYKQWLEGE TGCEFYAFCG ALFGISSMIT | |
| | LTAIALDRYL VITRPLATFG VASKRRAAFV LLGVWLYALA | |
| | WSLPPFFGWS AYVPEGLLTS CSWDYMSFTP AVRAYTMLLC | |
| | CFVFFLPLLI IIYCYIFIFR AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRQKF RVTFLKIWND HFRCQPAPPI | |
| | DEDLPEERPD D | |
| P383A | WT human melanopsin (SEQ ID NO: 1) engineered to have P383A | |
| | substitution. | |
| P383D | WT human melanopsin (SEQ ID NO: 1) engineered to have P383D | |
| | substitution. | |
| Y382S | WT human melanopsin (SEQ ID NO: 1) engineered to have Y382S | |
| | substitution. | |
| Y382D | WT human melanopsin (SEQ ID NO: 1) engineered to have Y382D | |
| | substitution. | |
| S384A | WT human melanopsin (SEQ ID NO: 1) engineered to have S384A | |
| | substitution. | |
| S384D | WT human melanopsin (SEQ ID NO: 1) engineered to have S384D | |
| | substitution. | |
| S384R | WT human melanopsin (SEQ ID NO: 1) engineered to have S384R | |
| | substitution. | |
| T129S | WT human melanopsin (SEQ ID NO: 1) engineered to have T129S | |
| | substitution. | |
| T129D | WT human melanopsin (SEQ ID NO: 1) engineered to have T129D | |
| | substitution. | |
| Q135N | WT human melanopsin (SEQ ID NO: 1) engineered to have Q135N | |
| | substitution. | |
| M226T | WT human melanopsin (SEQ ID NO: 1) engineered to have M226T | |
| | substitution. | |
| M226S | WT human melanopsin (SEQ ID NO: 1) engineered to have M226S | |
| | substitution. | |
| E215H | WT human melanopsin (SEQ ID NO: 1) engineered to have E215H | |
| | substitution. | |
| E215S | WT human melanopsin (SEQ ID NO: 1) engineered to have E215S | |
| | substitution. | |
| Y212F | WT human melanopsin (SEQ ID NO: 1) engineered to have Y212F | |
| | substitution. | |
| Y212A | WT human melanopsin (SEQ ID NO: 1) engineered to have Y212A | |
| | substitution. | |
| Y212S | WT human melanopsin (SEQ ID NO: 1) engineered to have Y212S | |
| | substitution. | |
| Y212L | WT human melanopsin (SEQ ID NO: 1) engineered to have Y212L | |
| | substitution. | |
| P10L | WT human melanopsin (SEQ ID NO: 1) engineered to have P10L | |
| | substitution. | |
| P10A | WT human melanopsin (SEQ ID NO: 1) engineered to have P10A | |
| | substitution. | |
| P10G | WT human melanopsin (SEQ ID NO: 1) engineered to have P10G | |
| | substitution. | |
| P10F | WT human melanopsin (SEQ ID NO: 1) engineered to have P10F | |
| | substitution. | |
| T83M | WT human melanopsin (SEQ ID NO: 1) engineered to have T83M | |
| | substitution. | |
| T83L | WT human melanopsin (SEQ ID NO: 1) engineered to have T83L | |
| | substitution. | |
| T83A | WT human melanopsin (SEQ ID NO: 1) engineered to have T83A | |
| | substitution. | |
| T83S | WT human melanopsin (SEQ ID NO: 1) engineered to have T83S | |
| | substitution. | |
| S183A | WT human melanopsin (SEQ ID NO: 1) engineered to have S183A | |
| | substitution. | |
| N399D | WT human melanopsin (SEQ ID NO: 1) engineered to have N399D | |
| | substitution. | |
| R390A | WT human melanopsin (SEQ ID NO: 1) engineered to have R390A | |
| | substitution. | |
| R390D | WT human melanopsin (SEQ ID NO: 1) engineered to have R390D | |
| | substitution. | |
| H389S | WT human melanopsin (SEQ ID NO: 1) engineered to have H389S | |
| | substitution. | |
| R386A | WT human melanopsin (SEQ ID NO: 1) engineered to have R386A | |
| | substitution. | |
| R386D | WT human melanopsin (SEQ ID NO: 1) engineered to have R386D | |
| | substitution. | |
| P10F (419AA) | Truncated melanopsin variant, amino acids 1-419 of WT human | 5 |
| | melanopsin (SEQ ID NO: 3) engineered to have P10F substitution. | |
| T83L (419AA) | Truncated melanopsin variant, amino acids 1-419 of WT human | 6 |
| | melanopsin (SEQ ID NO: 3) engineered to have T83L substitution | |
| T129S (419AA) | Truncated melanopsin variant, amino acids 1-419 of WT human | 7 |
| | melanopsin (SEQ ID NO: 3) engineered to have T129S substitution | |
| Q135N (149AA) | Truncated melanopsin variant, amino acids 1-419 of WT human | 8 |
| | melanopsin (SEQ ID NO: 3) engineered to have Q135N substitution | |
| S183A (419AA) | Truncated melanopsin variant, amino acids 1-419 of WT human | 10 |
| | melanopsin (SEQ ID NO: 3) engineered to have S183A substitution | |
| Y212F (419AA) | Truncated melanopsin variant, amino acids 1-419 of WT human | 13 |
| | melanopsin (SEQ ID NO: 3) engineered to have Y212F substitution | |
| M226T (419AA) | Truncated melanopsin variant, amino acids 1-419 of WT human | 14 |
| | melanopsin (SEQ ID NO: 3) engineered to have M226T substitution | |
| Y382D (419AA) | Truncated melanopsin variant, amino acids 1-419 of WT human | 16 |
| | melanopsin (SEQ ID NO: 3) engineered to have Y382D substitution | |
| S384D (419AA) | Truncated melanopsin variant, amino acids 1-419 of WT human | 17 |
| | melanopsin (SEQ ID NO: 3) engineered to have S384D substitution | |
| R386A (419AA) | Truncated melanopsin variant, amino acids 1-419 of WT human | 18 |
| | melanopsin (SEQ ID NO: 3) engineered to have R386A substitution | |
| R390D (419AA) | Truncated melanopsin variant, amino acids 1-419 of WT human | 20 |
| | melanopsin (SEQ ID NO: 3) engineered to have R390D substitution | |
| P10F, T83M | Truncated melanopsin variant, amino acids 1-419 of WT human | |
| | melanopsin (SEQ ID NO: 3) engineered to have P10F and T83M | |
| | substitutions. | |
| Q135N, S183A | Truncated melanopsin variant, amino acids 1-419 of WT human | 11 |
| | melanopsin (SEQ ID NO: 3) engineered to have Q135N and S183A | |
| | substitutions. | |
| Y212F, M226T | Truncated melanopsin variant, amino acids 1-419 of WT human | 15 |
| | melanopsin (SEQ ID NO: 3) engineered to have Y212F and M226T | |
| | substitutions. | |
| T129S, Q135N | Truncated melanopsin variant, amino acids 1-419 of WT human | 9 |
| | melanopsin (SEQ ID NO: 3) engineered to have T129S and Q135N | |
| | substitutions. | |
| T129S, Q135N, | Truncated melanopsin variant, amino acids 1-419 of WT human | 12 |
| S138A | melanopsin (SEQ ID NO: 3) engineered to have T129S, Q135N, and | |
| | S138A substitutions. | |
| S138A, S384D, | Truncated melanopsin variant, amino acids 1-419 of WT human | 19 |
| R386A | melanopsin (SEQ ID NO: 3) engineered to have S138A, S384D, and | |
| (Also referred to | R386A substitutions. | |
| as | MNPPSGPRVP PSPTQEPSCM ATPAPPSWWD SSQSSISSLG | |
| “419AA S183A- | RLPSISPTAP GTWAAAWVPL PTVDVPDHAH YTLGTVILLV | |
| _S384D_R386A”) | GLTGMLGNLT VIYTFCRSRS LRTPANMFII NLAVSDELMS | |
| | FTQAPVFFTS SLYKQWLFGE TGCEFYAFCG ALFGISSMIT | |
| | LTAIALDRYL VITRPLATFG VAAKRRAAFV LLGVWLYALA | |
| | WSLPPFFGWS AYVPEGLLTS CSWDYMSFTP AVRAYTMLLC | |
| | CFVFFLPLLI IIYCYIFIER AIRETGRALQ TFGACKGNGE | |
| | SLWQRQRLQS ECKMAKIMLL VILLFVLSWA PYSAVALVAF | |
| | AGYAHVLTPY MSSVPAVIAK ASAIHNPIIY AITHPKYRVA | |
| | IAQHLPCLGV LLGVSRRHSR PYPDYASTHR STLTSHTSNL | |
| | SWISIRRRQE SLGSESEVG | |
| Y382D, R386A, | Truncated melanopsin variant, amino acids 1-419 of WT human | 21 |
| R390D | melanopsin (SEQ ID NO: 3) engineered to have Y382D, R386A, | |
| | and R390D substitutions. | |
| S138A, Y382D, | Truncated melanopsin variant, amino acids 1-419 of WT human | 22 |
| R386A, R390D | melanopsin (SEQ ID NO: 3) engineered to have S138A, Y382D, | |
| | R386A, and R390D substitutions. | |
| Y382D, S384D | Truncated melanopsin variant, amino acids 1-419 of WT human | |
| R386A, R390D | melanopsin (SEQ ID NO: 3) engineered to have Y382D, S384D | |
| | R386A, and R390D substitutions. | |
| T129S, S183A, | Truncated melanopsin variant, amino acids 1-419 of WT human | 23 |
| M226T, Y382D, | melanopsin (SEQ ID NO: 3) engineered to have T129S, S183A, | |
| R386A, R390D | M226T, Y382D, R386A, and R390D substitutions. | |
| P10F, T83M, | Truncated melanopsin variant, amino acids 1-419 of WT human | |
| T129S, Q125N, | melanopsin (SEQ ID NO: 3) engineered to have P10F, T83M, | |
| S183A, Y212F, | T129S, Q125N, S183A, Y212F, and M226T substitutions. | |
| M226T | | |
| T129S, S183A, | Truncated melanopsin variant, amino acids 1-419 of WT human | |
| M226T, Y382D, | melanopsin (SEQ ID NO: 3) engineered to have T129S, S183A, | |
| S384D, R386A, | M226T, Y382D, S384D, R386A, and R390D substitutions. | |
| R390D | | |
| T83M, T129S, | Truncated melanopsin variant, amino acids 1-419 of WT human | |
| Q135N, S183A, | melanopsin (SEQ ID NO: 3) engineered to have T83M, T129S, | |
| Y212F, M226T, | Q135N, S183A, Y212F, M226T, Y382D, R386A, and R390D | |
| Y382D, R386A, | substitutions. | |
| R390D | | |
| T83M, T129S, | Truncated melanopsin variant, amino acids 1-419 of WT human | |
| Q135N, S183A, | melanopsin (SEQ ID NO: 3) engineered to have T83M, T129S, | |
| Y212F, M226T, | Q135N, S183A, Y212F, M226T, Y382D, S384D, R386A, and | |
| Y382D, S384D, | R390D substitutions. | |
| R386A, R390D | | |
| P10F, T83M, | Truncated melanopsin variant, amino acids 1-419 of WT human | |
| T129S, Q135N, | melanopsin (SEQ ID NO: 3) engineered to have P10F, T83M, | |
| S183A, Y212F, | T129S, Q135N, S183A, Y212F, M226T, Y382D, S384D, R386A, | |
| M226T, Y382D, | and R390D substitutions. | |
| S384D, R386A, | | |
| R390D | | |
| V370-R377Del | Variant in which amino acids 370-377 of WT melanopsin have been | |
| | removed. | |
| K356-R377Del | Variant in which amino acids 356-377 of WT melanopsin have been | |
| | removed. | |

The following experiments were performed to measure the amplitude/conductance light responses and the OFF light responses of the melanopsin variants shown in Table A above.

Briefly, HEK293T cells were transfected with (a) a nucleic acid encoding one melanopsin variant from Table A and either (b) a nucleic acid encoding GCaMP6s (an exemplary fluorescent calcium sensor described in Chen et al. (2013) Nature. 499 (7458): 295-300) or (c) a nucleic acid R-GECO1 (an exemplary fluorescent calcium sensor described in Zhao et al. (2011) Science. 333 (6051): 1888-1891). Parallel sets of transfections were performed using (a) a nucleic acid encoding wild-type melanopsin and either (b) a nucleic acid encoding GCaMP6s or (c) a nucleic acid R-GECO1. After 72 hours, cells were imaged in the ImageXpress micro confocal system (Molecular Devices) to record the change in fluorescence over time. To measure amplitude/conductance light ON responses, cells co-transfected with GCaMP6 were imaged under the FITC channel which enables recording from GCaMP6s calcium induced fluorescence while simultaneously activating melanopsin or melanopsin variant. To measure OFF light responses, cells co-transfected with R-GECO1 were briefly illuminated with 480 nm light to activate melanopsin or melanopsin variant before being recorded under the TexasRed channel to recording from R-GECO1 calcium induced fluorescence. The amplitude of the light response from the ON assay was calculated from the starting baseline recording point to the greatest fluorescence achieved. The mean lifetime of decay, TauOFF, from the OFF assay was calculated from the inversion of the rate constant K, which was determined by the first order rate law from the decay of measured fluorescence over time.

In the ON assay, the melanopsin or melanopsin variant light induced flux of intracellular calcium resulted in fluorescence from GCaMP6s. Light induced fluorescence increased logarithmically over time. The amplitude of the calcium-induced fluorescence was correlated to the light sensitivity of the WT human melanopsin or the melanopsin variant. Melanopsin variants that had a greater amplitude/light sensitivity were identified.

The OFF assay measures the cessation of the WT human melanopsin or melanopsin variant light response, with the depletion of intracellular calcium exponentially decreasing R-GECO1 calcium induced fluorescence over time. Melanopsin variants that turned off faster, i.e., had a smaller TauOFF, than WT melanopsin were identified.

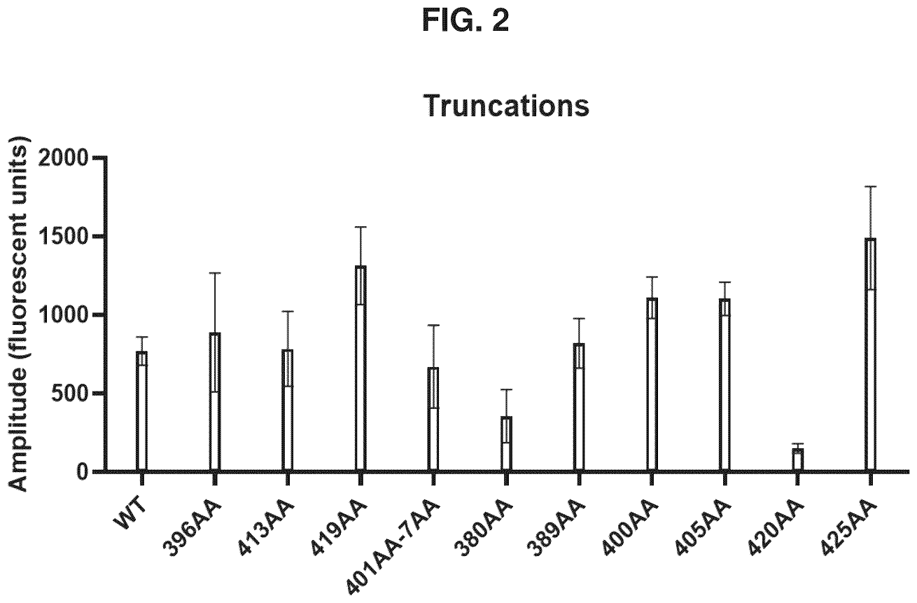

FIG. 2 and Table B show the results of experiments that were performed to determine the amplitude of calcium light responses (“ON” light responses) from melanopsin truncation variants. In the amplitude assay (“ON” assay) using GCaMP6s, the difference between baseline and the peak of light induced fluorescence was used to determine the amplitude of the light response. Several truncation variants, including 419aa and 425aa, had greater amplitudes than wild type. Error bars=standard error of the mean (SEM).

TABLE B
Amplitude of Calcium Light Responses
from Melanopsin Truncation Variants

| | Amplitude | | |
|---|---|---|---|
| Variant | (fluorescent units) | Stdev | N |
| WT | 770 | 270 | 9 |
| 396AA | 890 | 850 | 5 |
| 413AA | 780 | 590 | 6 |
| 419AA | 1310 | 610 | 6 |
| 401AA-7AA | 670 | 590 | 5 |
| 380AA | 360 | 410 | 6 |
| 389AA | 820 | 480 | 9 |
| 400AA | 1110 | 390 | 9 |
| 405AA | 1100 | 320 | 9 |
| 420AA | 150 | 90 | 9 |
| 425AA | 1490 | 990 | 9 |

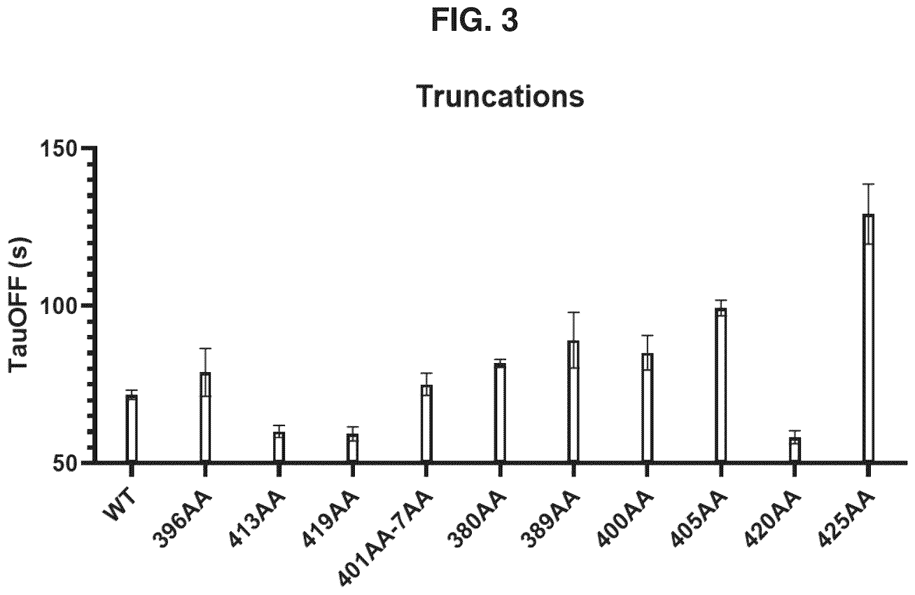

FIG. 3 and Table C show the results of experiments that were performed to determine the TauOFF of calcium light responses from melanopsin truncation variants. In the OFF assay using RGECO-1, the rate constant k for the decay of the induced fluorescent light response was used to find the mean time of decay, TauOFF. Several truncation variants, including 413AA, 419AA, and 420AA, had a lower TauOFF than wild type. Error bars=SEM

TABLE C
TauOFF of Calcium Light Responses
from Melanopsin Truncation Variants

| Variant | TauOFF (s) | StDev | N |
|---|---|---|---|
| WT | 72 | 4 | 9 |
| 396AA | 79 | 23 | 9 |
| 413AA | 60 | 6 | 9 |
| 419AA | 59 | 6 | 6 |
| 401AA-7AA | 75 | 10 | 9 |
| 380AA | 82 | 3 | 5 |
| 389AA | 89 | 18 | 4 |
| 400AA | 85 | 17 | 9 |
| 405AA | 99 | 8 | 9 |
| 420AA | 58 | 6 | 9 |
| 425AA | 129 | 29 | 9 |

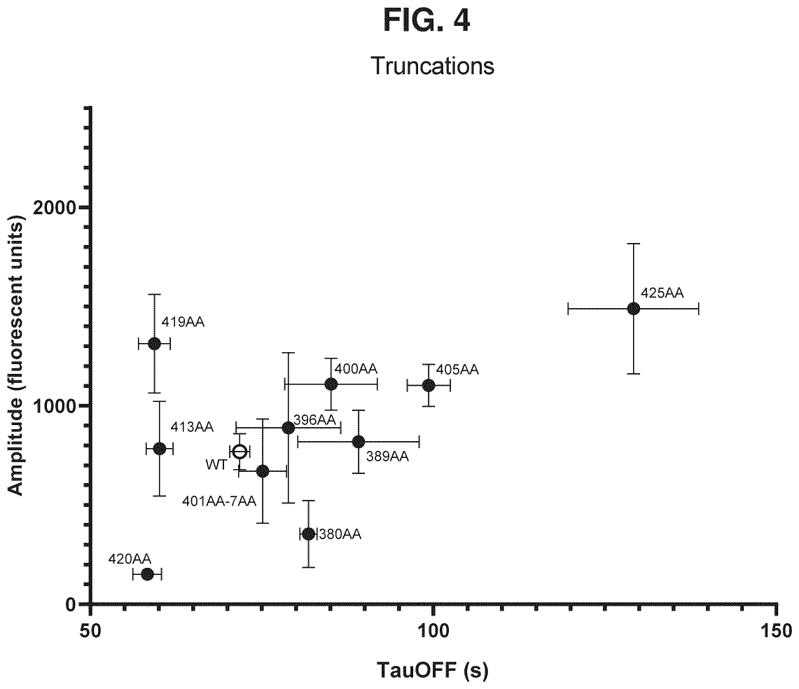

FIG. 4 shows a comparison of the amplitude versus the TauOFF for the truncation variants shown in FIGS. 2 and 3. Variants (black circles) like 419AA have both a smaller TauOFF and greater amplitude than wild type (white circle). Error bars=SEM.

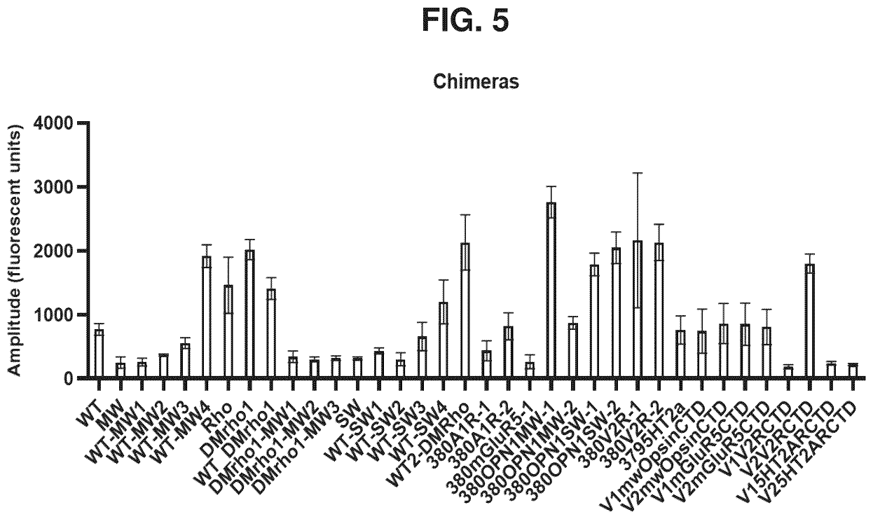

FIG. 5 and Table D show the results of experiments that were performed to determine the amplitude of calcium light responses from chimeric melanopsin variants. In the ON assay using GCaMP6s, the difference between baseline and the peak of light induced fluorescence was used to determine the amplitude of the light response. Error bars=SEM.

TABLE D
Amplitude of Calcium Light Responses
from Chimeric Melanopsin Variants

| | Amplitude | | |
|---|---|---|---|
| Variant | (fluorescent units) | Stdev | N |
| WT | 770 | 270 | 9 |
| MW | 250 | 220 | 6 |
| WT-MW1 | 260 | 150 | 6 |
| WT-MW2 | 370 | 32 | 6 |
| WT-MW3 | 550 | 210 | 6 |
| WT-MW4 | 1920 | 440 | 6 |
| Rho | 1460 | 1080 | 6 |
| DMrho1 | 2020 | 390 | 6 |
| WT_DMrho1 | 1410 | 420 | 6 |
| DMrho1-MW1 | 340 | 230 | 6 |
| DMrho1-MW2 | 300 | 130 | 9 |
| DMrho1-MW3 | 320 | 120 | 9 |
| SW | 320 | 77 | 9 |
| WT-SW1 | 430 | 69 | 2 |
| WT-SW2 | 300 | 310 | 9 |
| WT-SW3 | 660 | 550 | 6 |
| WT-SW4 | 1200 | 1040 | 9 |
| WT2-DMRho | 2130 | 1150 | 7 |
| 380A1R-1 | 430 | 440 | 8 |
| 380A1R-2 | 820 | 600 | 8 |
| 380mGluR5-1 | 270 | 310 | 8 |
| 380OPN1MW-1 | 2760 | 610 | 6 |
| 380OPN1MW-2 | 870 | 290 | 9 |
| 380OPN1SW-1 | 1790 | 540 | 9 |
| 380OPN1SW-2 | 2050 | 750 | 9 |
| 380V2R-1 | 2170 | 3170 | 9 |
| 380V2R-2 | 2130 | 850 | 9 |
| 3795HT2a | 760 | 670 | 9 |
| V1mwOpsinCTD | 740 | 780 | 5 |
| V2mwOpsinCTD | 860 | 760 | 6 |
| V1mGluR5CTD | 850 | 1000 | 9 |
| V2mGluR5CTD | 810 | 830 | 9 |
| V1V2RCTD | 180 | 99 | 9 |
| V2V2RCTD | 1800 | 460 | 9 |
| V15HT2ARCTD | 240 | 96 | 9 |
| V25HT2ARCTD | 220 | 60 | 9 |

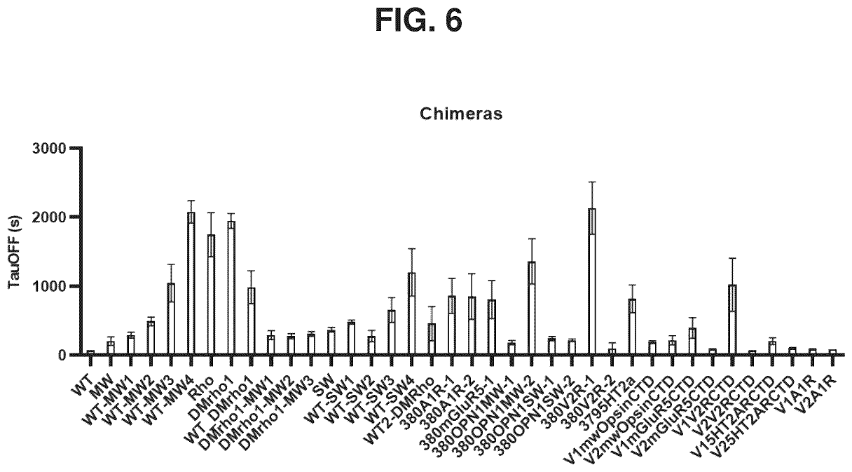

FIG. 6 and Table E show the results of experiments that were performed to determine the TauOFF of calcium light responses from chimeric melanopsin variants. In the OFF assay using RGECO-1, the rate constant k for the decay of the induced fluorescent light response was used to find the mean time of decay, TauOFF. Error bars=SEM.

TABLE E
TauOFF of Calcium Light Responses
from Melanopsin Chimeric Variants

| Variant | TauOFF (s) | Stdev | N |
|---|---|---|---|
| WT | 72 | 4 | 9 |
| MW | 232 | 17 | 6 |
| WT-MW1 | 278 | 295 | 9 |
| WT-MW2 | 194 | 69 | 8 |
| WT-MW3 | 138 | 37 | 8 |
| WT-MW4 | 77 | 16 | 8 |
| Rho | 106 | 47 | 8 |
| DMrho1 | 182 | 91 | 9 |
| WT_DMrho1 | 90 | 21 | 9 |
| DMrho1-MW1 | 273 | 73 | 9 |
| DMrho1-MW2 | 457 | 490 | 9 |
| DMrho1-MW3 | 559 | 477 | 9 |
| SW | 129 | 65 | 9 |
| WT-SW1 | 131 | 71 | 9 |
| WT-SW2 | 119 | 60 | 9 |
| WT-SW3 | 89 | 19 | 9 |
| WT-SW4 | 86 | 5 | 6 |
| WT2-DMRho | 71 | 4 | 5 |
| 380A1R-1 | 74 | 3 | 6 |
| 380A1R-2 | 77 | 9 | 6 |
| 380mGluR5-1 | 71 | 5 | 6 |
| 380OPN1MW-1 | 77 | 7 | 6 |
| 380OPN1MW-2 | 98 | 10 | 6 |
| 380OPN1SW-1 | 75 | 12 | 6 |
| 380OPN1SW-2 | 80 | 24 | 6 |
| 380V2R-1 | 70 | 10 | 5 |
| 380V2R-2 | 64 | 5 | 5 |
| 3795HT2a | 89 | 26 | 6 |
| V1mwOpsinCTD | 188 | 69 | 9 |
| V2mwOpsinCTD | 215 | 206 | 9 |
| V1mGluR5CTD | 393 | 366 | 6 |
| V2mGluR5CTD | 87 | 19 | 9 |
| V1V2RCTD | 1018 | 1020 | 7 |
| V2V2RCTD | 63 | 9 | 9 |
| V15HT2ARCTD | 204 | 132 | 7 |
| V25HT2ARCTD | 100 | 36 | 9 |
| V1A1R | 90 | 11 | 6 |
| V2A1R | 85 | 7 | 9 |

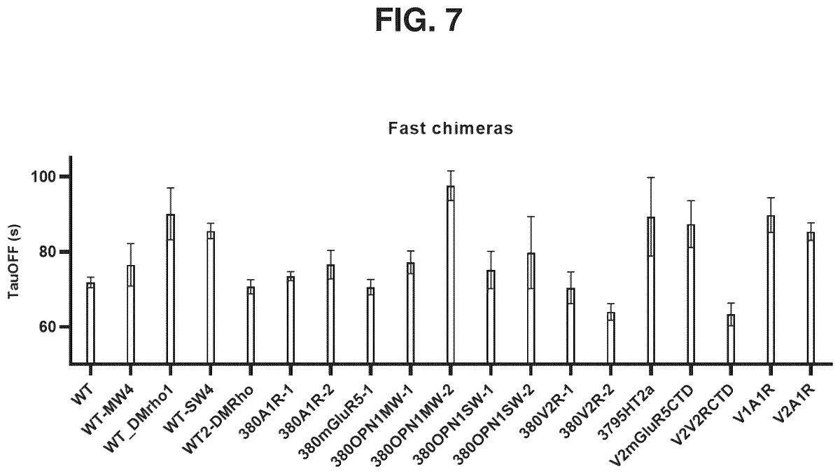

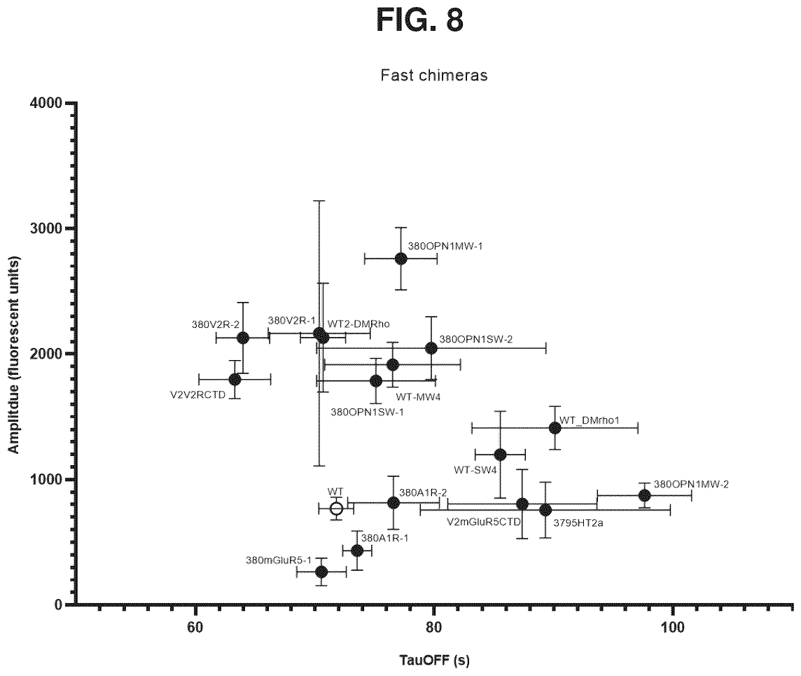

FIG. 7 and Table F show the results experiments that were performed to determine the TauOFF of calcium light responses from the fastest chimeric melanopsin variants shown in FIG. 6. In the OFF assay using RGECO-1, the rate constant k for the decay of the induced fluorescent light response was used to find the mean time of decay, TauOFF. Error bars=SEM. FIG. 8 shows a comparison of the amplitude of the ON response versus the TauOFF for the fastest chimeric melanopsin variants shown in FIG. 7 and Table F. Variants are black circles and wild type is a white circle.

TABLE F
TauOFF of Calcium Light Responses from the fastest
Melanopsin Chimeric Variants from Table E

| Variant | TauOFF (s) | Stdev | N |
|---|---|---|---|
| WT | 72 | 4 | 9 |
| WT-MW4 | 77 | 16 | 8 |
| WT_DMrho1 | 90 | 21 | 9 |
| WT-SW4 | 86 | 5 | 6 |
| WT2-DMRho | 71 | 4 | 5 |
| 380A1R-1 | 74 | 3 | 6 |
| 380A1R-2 | 77 | 9 | 6 |
| 380mGluR5-1 | 71 | 5 | 6 |
| 380OPN1MW-1 | 77 | 7 | 6 |
| 380OPN1MW-2 | 98 | 10 | 6 |
| 380OPN1SW-1 | 75 | 12 | 6 |
| 380OPN1SW-2 | 80 | 24 | 6 |
| 380V2R-1 | 70 | 10 | 5 |
| 380V2R-2 | 64 | 5 | 5 |
| 3795HT2a | 89 | 26 | 6 |
| V2mGluR5CTD | 87 | 19 | 9 |
| V2V2RCTD | 63 | 9 | 9 |
| V1A1R | 90 | 11 | 6 |
| V2A1R | 85 | 7 | 9 |

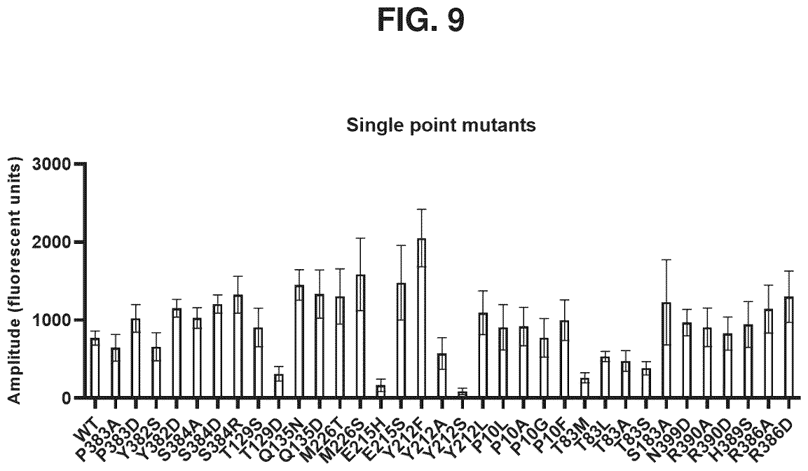

FIG. 9 and Table G show the results of experiments that were performed to determine the amplitude of calcium light responses from substituted full length melanopsin variants. In the ON assay using GCaMP6s, the difference between baseline and the peak of light induced fluorescence was used to determine the amplitude of the light response. Error bars=SEM.

TABLE G
Amplitude of Calcium Light Responses from Substituted
Full Length Melanopsin Variants.

| | Amplitude | | |
|---|---|---|---|
| Variant | (fluorescent units) | Stdev | N |
| WT | 770 | 270 | 9 |
| P383A | 650 | 520 | 9 |
| P383D | 1020 | 530 | 9 |
| Y382S | 660 | 540 | 9 |
| Y382D | 1150 | 340 | 9 |
| S384A | 1030 | 400 | 9 |
| S384D | 1210 | 350 | 9 |
| S384R | 1330 | 710 | 9 |
| T129S | 900 | 740 | 9 |
| T129D | 310 | 230 | 6 |
| Q135N | 1450 | 590 | 9 |
| Q135D | 1340 | 930 | 9 |
| M226T | 1300 | 1060 | 9 |
| M226S | 1590 | 1400 | 9 |
| E215H | 160 | 240 | 9 |
| E215S | 1480 | 1440 | 9 |
| Y212F | 2050 | 1110 | 9 |
| Y212A | 570 | 610 | 9 |
| Y212S | 86 | 120 | 9 |
| Y212L | 1100 | 840 | 9 |
| P10L | 910 | 870 | 9 |
| P10A | 920 | 740 | 9 |
| P10G | 770 | 740 | 9 |
| P10F | 1000 | 780 | 9 |
| T83M | 260 | 200 | 9 |
| T83L | 530 | 200 | 9 |
| T83A | 480 | 400 | 9 |
| T83S | 380 | 260 | 9 |
| S183A | 1230 | 1640 | 9 |
| N399D | 970 | 510 | 9 |
| R390A | 910 | 740 | 9 |
| R390D | 830 | 600 | 8 |
| H389S | 940 | 830 | 8 |
| R386A | 1140 | 870 | 8 |
| R386D | 1300 | 930 | 8 |

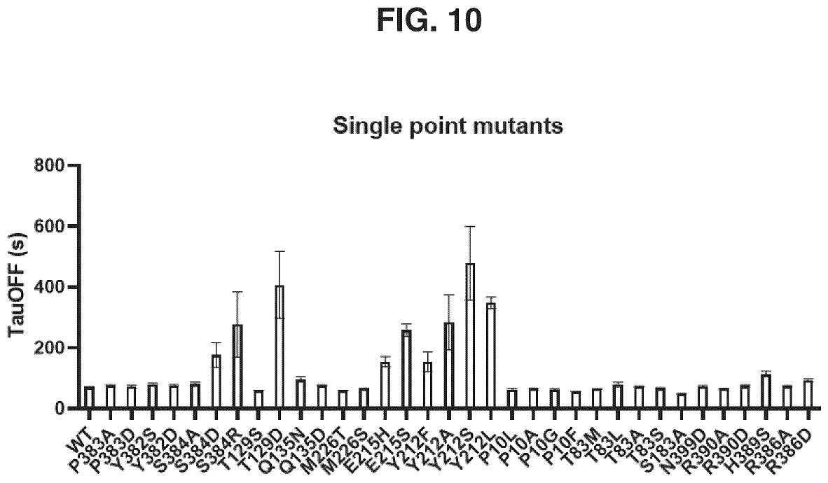

FIG. 10 and Table H show the results of experiments that were performed to determine the TauOFF of calcium light responses from substituted full length melanopsin variants. In the OFF assay using RGECO-1, the rate constant k for the decay of the induced fluorescent light response was used to find the mean time of decay, TauOFF. Error bars=SEM.

TABLE H
TauOFF of Calcium Light Responses from Substituted
Full Length Melanopsin Variants

| Variant | TauOFF (s) | Stdev | N |
|---|---|---|---|
| WT | 72 | 4 | 9 |
| P383A | 76 | 11 | 9 |
| P383D | 73 | 14 | 9 |
| Y382S | 79 | 14 | 9 |
| Y382D | 76 | 10 | 9 |
| S384A | 81 | 18 | 6 |
| S384D | 100 | 16 | 6 |
| S384R | 73 | 10 | 6 |
| T129S | 60 | 2 | 9 |
| T129D | 276 | 116 | 9 |
| Q135N | 96 | 26 | 9 |
| Q135D | 76 | 9 | 9 |
| M226T | 60 | 3 | 9 |
| M226S | 65 | 8 | 8 |
| E215H | 155 | 51 | 9 |
| E215S | 259 | 50 | 6 |
| Y212F | 154 | 80 | 6 |
| Y212A | 193 | 28 | 9 |
| Y212S | 479 | 365 | 9 |
| Y212L | 348 | 47 | 6 |
| P10L | 64 | 9 | 9 |
| P10A | 65 | 10 | 9 |
| P10G | 64 | 7 | 9 |
| P10F | 56 | 2 | 9 |
| T83M | 65 | 2 | 9 |
| T83L | 80 | 18 | 6 |
| T83A | 73 | 6 | 9 |
| T83S | 68 | 6 | 9 |
| S183A | 49 | 4 | 9 |
| N399D | 73 | 11 | 9 |
| R390A | 65 | 9 | 9 |
| R390D | 75 | 9 | 9 |
| H389S | 115 | 29 | 9 |
| R386A | 74 | 7 | 9 |
| R386D | 93 | 18 | 9 |

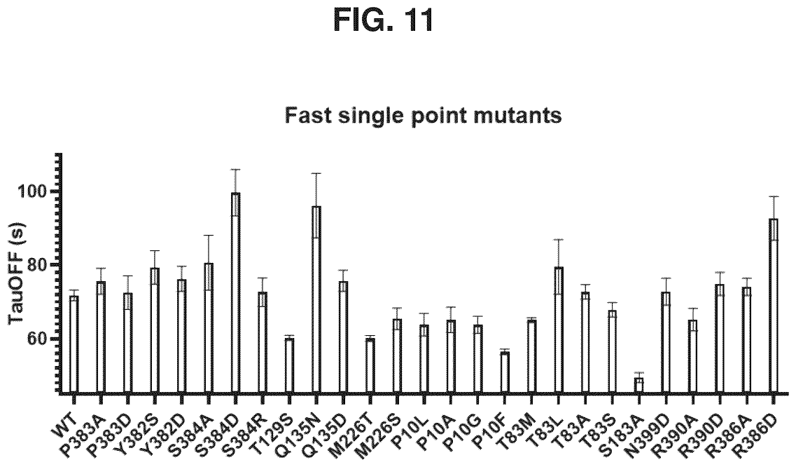

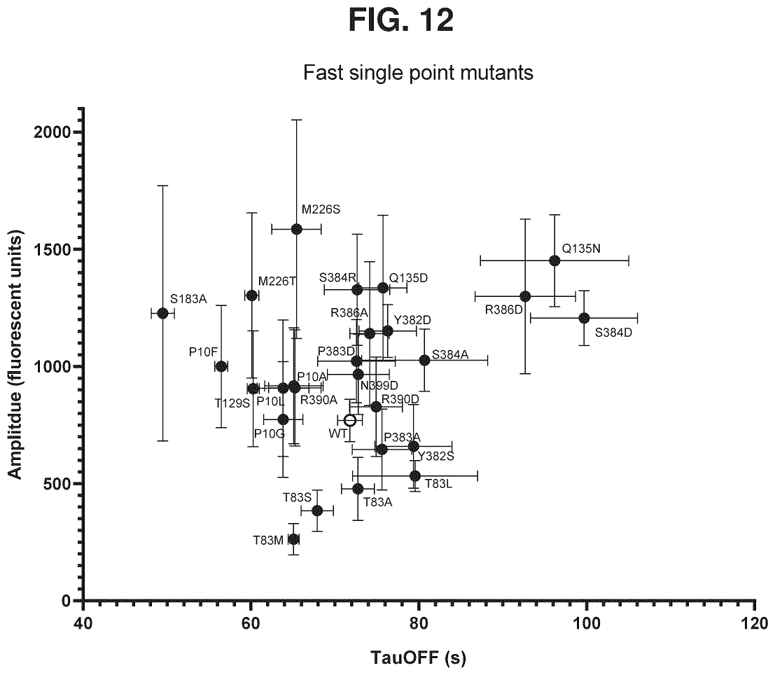

FIG. 11 and Table I show the results of experiments that were performed to determine the TauOFF of calcium light responses from the fastest substituted full length melanopsin variants shown in FIG. 10. In the OFF assay using RGECO-1, the rate constant k for the decay of the induced fluorescent light response was used to find the mean time of decay, TauOFF. Error bars=SEM. FIG. 12 shows a comparison of the amplitude of the ON response versus the TauOFF for the fastest substituted full length melanopsin variants in FIG. 11. Variants are black circles and wild type is a white circle. Error bars=SEM

TABLE I
TauOFF of Calcium Light Responses from the fastest Substituted
Full Length Melanopsin Variants from Table H

| Variant | TauOFF (s) | Stdev | N |
|---|---|---|---|
| WT | 72 | 4 | 9 |
| P383A | 76 | 11 | 9 |
| P383D | 73 | 14 | 9 |
| Y382S | 79 | 14 | 9 |
| Y382D | 76 | 10 | 9 |
| S384A | 81 | 18 | 6 |
| S384D | 100 | 16 | 6 |
| S384R | 73 | 10 | 6 |
| T129S | 60 | 2 | 9 |
| Q135N | 96 | 26 | 9 |
| Q135D | 76 | 9 | 9 |
| M226T | 60 | 3 | 9 |
| M226S | 65 | 8 | 8 |
| P10L | 64 | 9 | 9 |
| P10A | 65 | 10 | 9 |
| P10G | 64 | 7 | 9 |
| P10F | 56 | 2 | 9 |
| T83M | 65 | 2 | 9 |
| T83L | 80 | 18 | 6 |
| T83A | 73 | 6 | 9 |
| T83S | 68 | 6 | 9 |
| S183A | 49 | 4 | 9 |
| N399D | 73 | 11 | 9 |
| R390A | 65 | 9 | 9 |
| R390D | 75 | 9 | 9 |
| R386A | 74 | 7 | 9 |
| R386D | 93 | 18 | 9 |

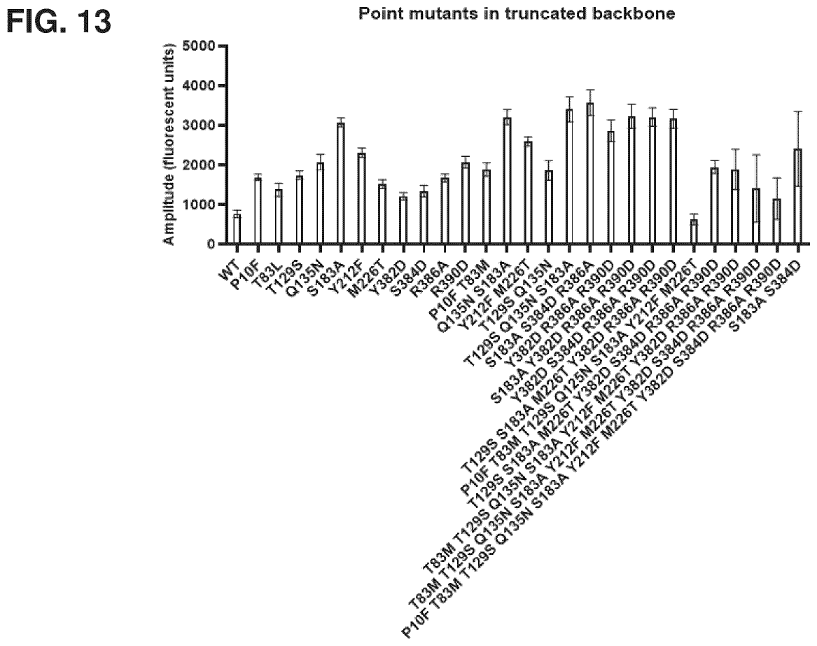

FIG. 13 and Table J shows the amplitude of calcium light responses from substituted melanopsin variants in a truncated backbone (419AA, SEQ ID NO: 3). In the assay using GCaMP6s, the difference between baseline and the peak of light induced fluorescence was used to determine the amplitude of the light response. Error bars=SEM.

TABLE J
Amplitude of Calcium Light Responses from Substituted
Melanopsin Variants in a Truncated Backbone

| | Amplitude | | |
|---|---|---|---|
| Variant | (fluorescent units) | Stdev | N |
| WT | 770 | 270 | 9 |
| P10F | 1690 | 240 | 9 |
| T83L | 1380 | 510 | 9 |
| T129S | 1740 | 320 | 9 |
| Q135N | 2070 | 600 | 9 |
| S183A | 3070 | 350 | 9 |
| Y212F | 2310 | 340 | 9 |
| M226T | 1510 | 340 | 9 |
| Y382D | 1220 | 280 | 9 |
| S384D | 1340 | 440 | 9 |
| R386A | 1670 | 290 | 9 |
| R390D | 2070 | 440 | 9 |
| P10F T83M | 1880 | 520 | 9 |
| Q135N S183A | 3200 | 570 | 9 |
| Y212F M226T | 2590 | 340 | 9 |
| T129S Q135N | 1860 | 750 | 9 |
| T129S Q135N S183A | 3400 | 930 | 9 |
| S183A S384D R386A | 3570 | 980 | 9 |
| Y382D R386A R390D | 2860 | 830 | 9 |
| S183A Y382D R386A R390D | 3230 | 900 | 9 |
| Y382D S384D R386A R390D | 3210 | 690 | 9 |
| T129S S183A M226T Y382D R386A | 3170 | 700 | 9 |
| R390D | | | |
| P10F T83M T129S Q125N S183A | 630 | 400 | 9 |
| Y212F M226T | | | |
| T129S S183A M226T Y382D S384D | 1940 | 500 | 9 |
| R386A R390D | | | |
| T83M T129S Q135N S183A Y212F | 1890 | 1530 | 9 |
| M226T Y382D R386A R390D | | | |
| T83M T129S Q135N S183A Y212F | 1410 | 2080 | 6 |
| M226T Y382D S384D R386A R390D | | | |
| P10F T83M T129S Q135N S183A | 1150 | 1480 | 8 |
| Y212F M226T Y382D S384D R386A | | | |
| R390D | | | |
| S183A S384D | 2400 | 2100 | 5 |

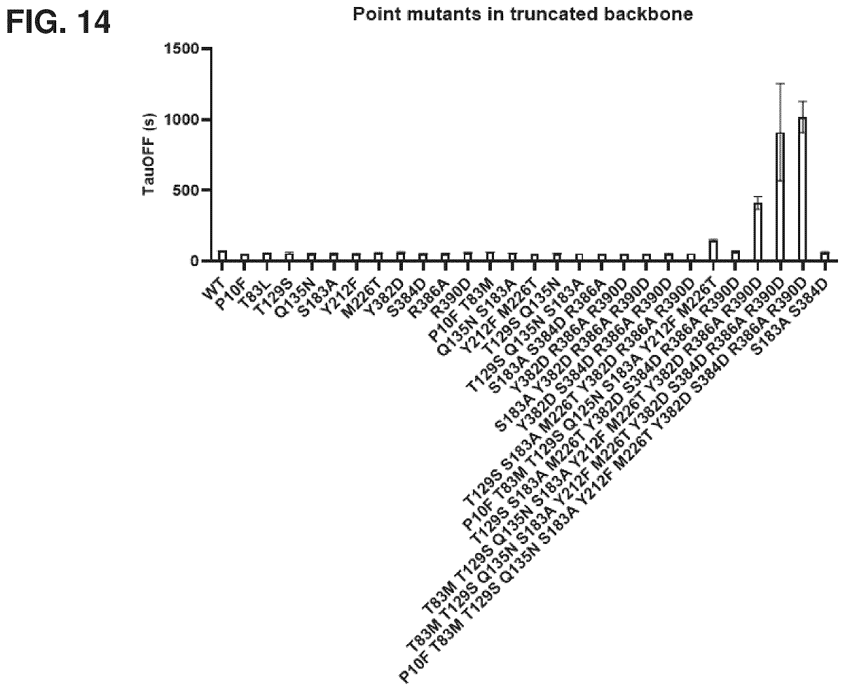

FIG. 14 and Table K show the results of experiments that were performed to determine the TauOFF of calcium light responses from substituted melanopsin variants in a truncated backbone (419AA, SEQ ID NO: 3). In the OFF assay using RGECO-1, the rate constant k for the decay of the induced fluorescent light response was used to find the mean time of decay, TauOFF. Error bars=SEM.

TABLE K
TauOFF of Calcium Light Responses from Substituted
Melanopsin Variants in a Truncated Backbone

| Variant | TauOFF(s) | Stdev | N |
|---|---|---|---|
| WT | 72 | 4 | 9 |
| P10F | 48 | 1 | 3 |
| T83L | 57 | 3 | 6 |
| T129S | 55 | 18 | 7 |
| Q135N | 53 | 5 | 6 |
| S183A | 53 | 12 | 5 |
| Y212F | 52 | 4 | 6 |
| M226T | 58 | 6 | 6 |
| Y382D | 62 | 12 | 6 |
| S384D | 52 | 2 | 5 |
| R386A | 53 | 3 | 6 |
| R390D | 58 | 9 | 6 |
| P10F T83M | 63 | 4 | 6 |
| Q135N S183A | 56 | 3 | 6 |
| Y212F M226T | 52 | 2 | 6 |
| T129S Q135N | 55 | 2 | 6 |
| T129S Q135N S183A | 56 | 1 | 6 |
| S183A S384D R386A | 48 | 1 | 6 |
| Y382D R386A R390D | 52 | 1 | 6 |
| S183A Y382D R386A R390D | 51 | 1 | 6 |
| Y382D S384D R386A R390D | 52 | 2 | 5 |
| T129S S183A M226T Y382D R386A R390D | 55 | 2 | 6 |
| P10F T83M T129S Q125N S183A Y212F | 145 | 21 | 5 |
| M226T | | | |
| T129S S183A M226T Y382D S384D R386A | 71 | 6 | 5 |
| R390D | | | |
| T83M T129S Q135N S183A Y212F M226T | 411 | 110 | 6 |
| Y382D R386A R390D | | | |
| T83M T129S Q135N S183A Y212F M226T | 911 | 685 | 4 |
| Y382D S384D R386A R390D | | | |
| P10F T83M T129S Q135N S183A Y212F | 1019 | 221 | 4 |
| M226T Y382D S384D R386A R390D | | | |
| S183A S384D | 62 | 14 | 6 |

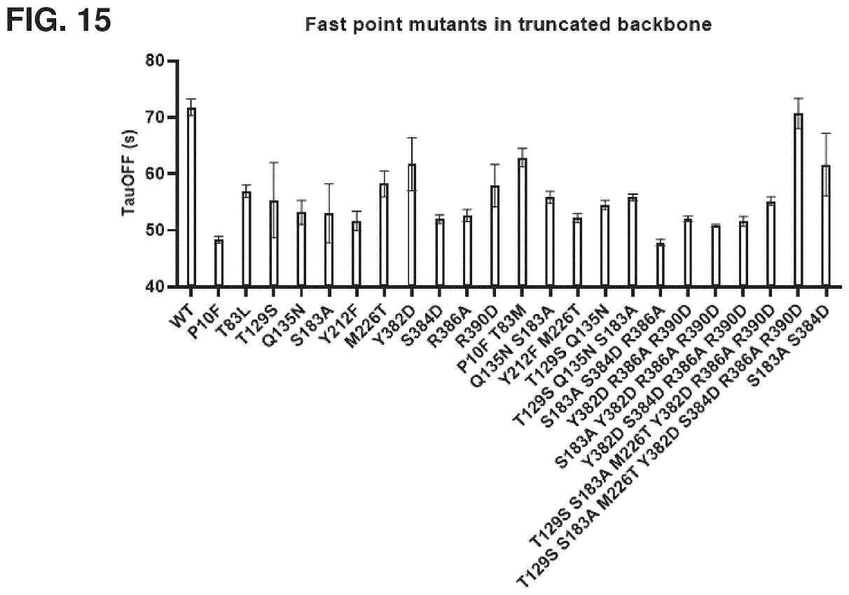

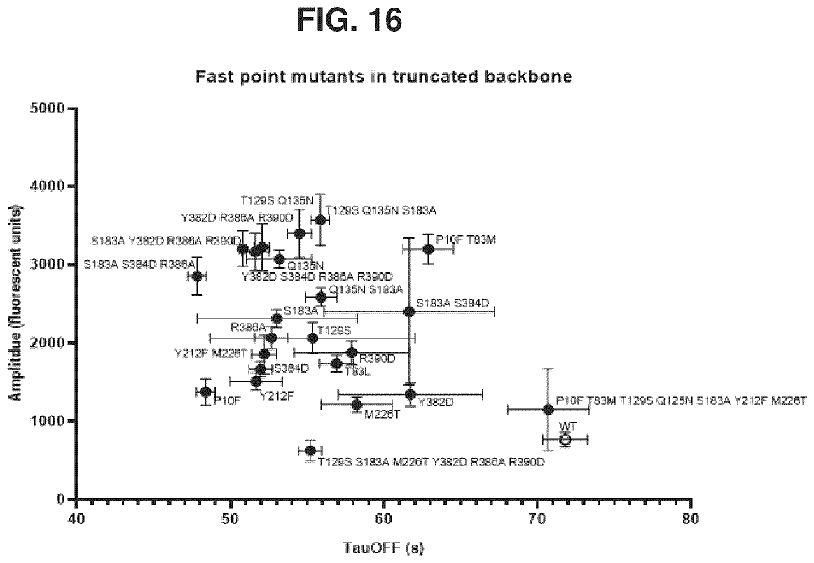

FIG. 15 and Table L show the results of experiments that were performed to determine the TauOFF of calcium light responses from the fastest substituted melanopsin variants in a truncated backbone shown in FIG. 14. In the OFF assay using RGECO-1, the rate constant k for the decay of the induced fluorescent light response was used to find the mean time of decay, TauOFF. Error bars=SEM. FIG. 16 shows a comparison of the amplitude of the ON response versus the TauOFF for the fastest substituted full length melanopsin variants in FIG. 14. Variants are black circles and wild type is a white circle. Error bars=SEM.

TABLE L
TauOFF of Calcium Light Responses from the Fastest Substituted
Melanopsin Variants in a Truncated Backbone from Table K

| Variant | TauOFF (s) | Stdev | N |
|---|---|---|---|
| WT | 72 | 4 | 9 |
| P10F | 48 | 1 | 3 |
| T83L | 57 | 3 | 6 |
| T129S | 55 | 18 | 7 |
| Q135N | 53 | 5 | 6 |
| S183A | 53 | 12 | 5 |
| Y212F | 52 | 4 | 6 |
| M226T | 58 | 6 | 6 |
| Y382D | 62 | 12 | 6 |
| S384D | 52 | 2 | 5 |
| R386A | 53 | 3 | 6 |
| R390D | 58 | 9 | 6 |
| P10F T83M | 63 | 4 | 6 |
| Q135N S183A | 56 | 3 | 6 |
| Y212F M226T | 52 | 2 | 6 |
| T129S Q135N | 55 | 2 | 6 |
| T129S Q135N S183A | 56 | 1 | 6 |
| S183A S384D R386A | 48 | 1 | 6 |
| Y382D R386A R390D | 52 | 1 | 6 |
| S183A Y382D R386A R390D | 51 | 1 | 6 |
| Y382D S384D R386A R390D | 52 | 2 | 5 |
| T129S S183A M226T Y382D R386A R390D | 55 | 2 | 6 |
| T129S S183A M226T Y382D S384D R386A | 71 | 6 | 5 |
| R390D | | | |
| S183A S384D | 62 | 14 | 6 |

### Example 1B: Electrophysiological Characterization of Melanopsin Variants

Patch clamp experiments are performed to measure the ion activity of HEK293T cells expressing a melanopsin variant shown in Table A. Parallel experiments are performed using HEK293T cells expressing wild type melanopsin. Briefly, HEK293T cells are transfected with (a) a nucleic acid encoding wild-type melanopsin or a melanopsin variant and (b) a nucleic acid encoding G protein-gated inwardly rectifying K⁺ channel 1 F137S (GIRKI_{F137S}). Transfected cells are placed in an external recording solution of 110 mM NaCl, 30 mM KCl, 2 mM CaCl₂), 10 mM HEPES, 5 mM glucose (pH 7.4) and are recorded using high-potassium solution containing 120 mM KCl, 1.75 mM MgCl₂, 5.735 mM CaCl₂), 10 mM EGTA, 5 mM Na₂ATP, and 10 mM HEPES (pH 7.2) using glass pipettes. Cells are whole cell patch-clamped to using an HEKA amplifier. 10 mM Acetylcholine (as GIRK activator) and 5 mM BaCl 2 (full block) are applied using a gravity-driven perfusion system, and illumination was controlled using a DG-4 system (Sutter) in combination with excitation filters. Cells are illuminated with different wavelengths of light to find those wavelengths to which WT human melanopsin and the melanopsin variants are most sensitive (i.e., “sensitive wavelengths”). While illuminated at a “sensitive wavelength,” WT human melanopsin and the melanopsin variants are exposed to a range of light intensities (1E+10 ph/cm²/s-10E+14 ph/cm²/s) to identify the intensities at which WT human melanopsin and the melanopsin variants are most sensitive

### Example 2: In Vivo Characterization of Melanopsin Variants

The activities of WT human melanopsin and the melanopsin variants in Table A are characterized in a mouse model of retinal degeneration. In the C3H/HeJ (Rd1) mouse line, mice experience retinal degeneration by weaning age due to a mutation in PDEbeta, which encodes the β-subunit of rod photoreceptor cGMP phosphodiesterase. This degeneration is consistent with retinitis pigmentosa experienced by human individuals with a mutation in human PDEbeta. WT human melanopsin or a melanopsin variant under the ubiquitous CMV promoter is packaged into AAV2.7m8 and intravitreally injected into 40 day old Rd1 mice at about 1E+10 vg/eye. After 49-50 days visual acuity is measured by optokinetic reflex testing (OKR), retinal light response is determined by full-field electroretingram (ffERG), and cerebral light response is recorded by visually evoked potentials (VEP).

Although the foregoing disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

### Example 3: Further Characterization of the Amplitude/Conductance Light Responses and the OFF Light Responses of Melanopsin Variants

Melanopsin has many best-in-class features for optogenetic proteins, but the wildtype protein (sequence schematic shown in FIG. 1) has slow deactivation, preventing rapid successive reactivation. Table M describes the mutational strategies undertaken to make calcium mediating rapid opsin. Such strategies are also discussed elsewhere herein, including in Examples 1A and 1B and in the Detailed Description.

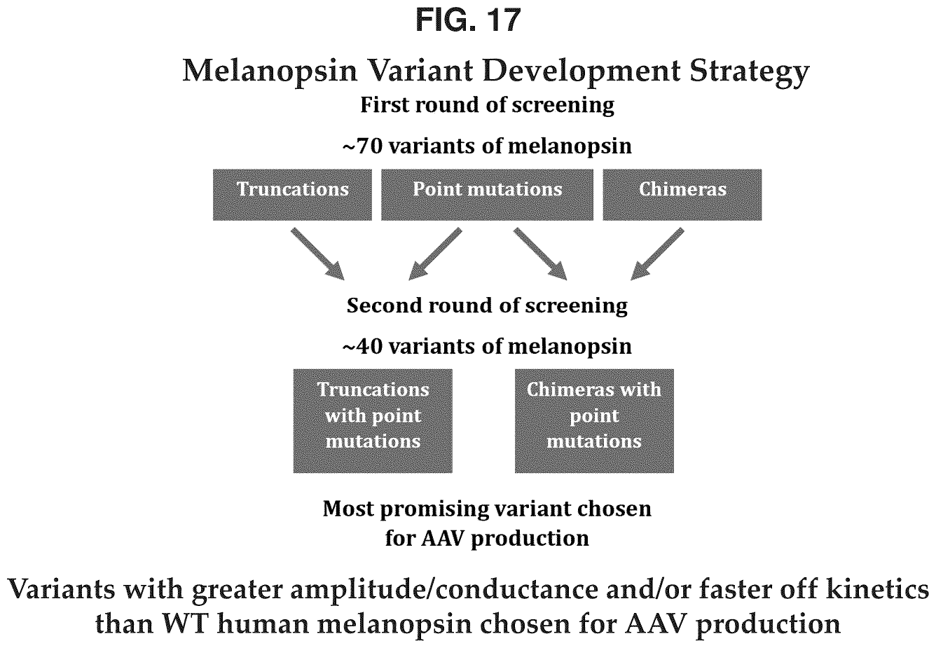

FIG. 17 shows a schematic of such melanopsin variant development strategy. About 70 melanopsin variants (see, e.g., Examples 1A and 1B and Tables A and M), comprising truncation variants, point mutation variants, and chimeras, were screened by the fluorescent calcium imaging assays in the first round of screening. The second round of screening identified about 40 melanopsin variants that were truncations or chimeras with synergistic point mutations. Melanopsin variants demonstrating increased amplitude and/or decreased TauOFF as compared to WT human melanopsin, e.g., according to the assays described in in Examples 1A and 1B and elsewhere herein, were chosen for AAV production.

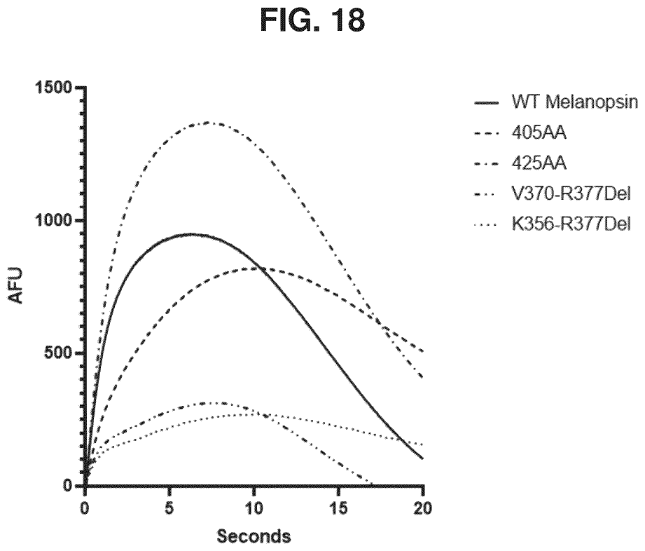

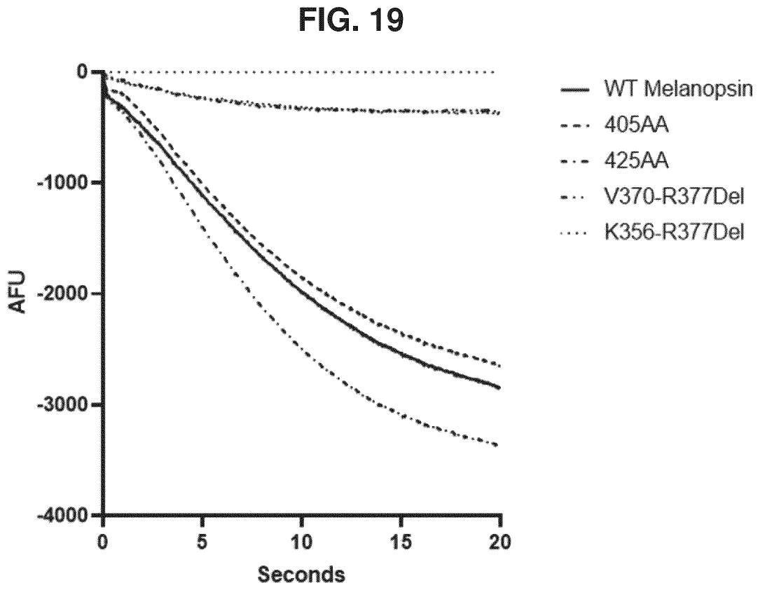

FIG. 18 shows the results of experiments that were performed to determine the amplitude of calcium light responses for WT human melanopsin, and exemplary melanopsin variants 405AA, 425AA, V370-R377Del, and K356-R377Del over time. FIG. 19 shows the results of experiments that were performed to determine the TauOFF of calcium light responses for wild type human melanopsin, exemplary melanopsin variants 405AA, 425AA, V370-R377Del, and K356-R377Del over time. In the amplitude assay (“ON” assay) using GCaMP6s, the difference between baseline and the peak of light induced fluorescence was used to determine the amplitude of the light response. In the OFF assay using RGECO-1, the rate constant k for the decay of the induced fluorescent light response was used to find the mean time of decay, TauOFF.

A selection of such mutations is noted on the schematic in FIG. 1. Some N-terminal melanopsin mutations increase speed and light sensitivity. Only one mutation in the transmembrane domains was successful. The transmembrane domains of GPCRs are is the most conserved. Loss of alcohol consistently improved amplitude and TauOFF. CTD chimeras required the first 377 amino acids of melanopsin to be successful. Many successful mutations in the putative phosphorylation and/or GRK/arrestin binding area were phosphomimics of amino acids normally phosphorylated during deactivation as well as mutations that reduced positive charge.

As noted above, FIG. 12 provides an exemplary comparison of the amplitude of the ON response versus the TauOFF for a subset of melanopsin variants shown in Table A. Error bars=standard error of the mean (SEM). Second round melanopsin variants included truncation variants and chimeric variants comprising synergistic point mutations. Melanopsin variants demonstrating both increased amplitudes and decreased TauOFF, such as 419AA_S183A_S384D_R386A (SEQ ID NO: 19), were selected for AAV production.

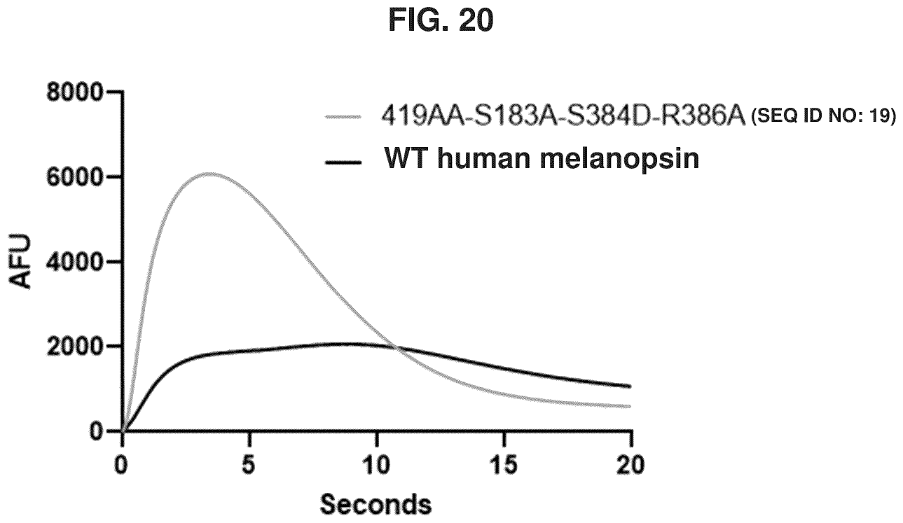

FIG. 20 shows the results of experiments that were performed to determine the amplitude of calcium light responses from cells transduced with AAV2.7m8 carrying WT human melanopsin or a melanopsin variant comprising SEQ ID NO 19.

(SEQ ID NO: 19)
MNPPSGPRVPPSPTQEPSCMATPAPPSWWDSSQSSISSLGRLPSISPTAP
GTWAAAWVPLPTVDVPDHAHYTLGTVILLVGLTGMLGNLTVIYTFCRSRS
LRTPANMFIINLAVSDFLMSFTQAPVFFTSSLYKQWLFGETGCEFYAFCG
ALFGISSMITLTAIALDRYLVITRPLATFGVAAKRRAAFVLLGVWLYALA
WSLPPFFGWSAYVPEGLLTSCSWDYMSFTPAVRAYTMLLCCFVFFLPLLI
IIYCYIFIFRAIRETGRALQTFGACKGNGESLWQRQRLQSECKMAKIMLL
VILLFVLSWAPYSAVALVAFAGYAHVLTPYMSSVPAVIAKASAIHNPIIY
AITHPKYRVAIAQHLPCLGVLLGVSRRHSRPYPDYASTHRSTLTSHTSNL
SWISIRRRQESLGSESEVG

SEQ ID NO: 19 comprises amino acids 1-419 of WT human melanopsin (SEQ ID NO: 3) and has been engineered to comprise S183A, S384D, and R386A substitutions (see Table A).

AAV2.7m8-CMV-WT human melanopsin and AAV2.7m8-CMV-SEQ ID NO: 19 were produced using AAVMAX suspension cells, AVB Column, CsCl ultracentrifugation, and buffer exchange. The resulting rAAVs were analyzed via ddPCR titer, Western blot, silver stain, and alkaline gel.

HEK293T cells were transduced with AAV2.7m8-CMV-SEQ ID NO: 19 or AAV2.7m8-CMV-WT human melanopsin and with AAV2.7m8-CMV-GCAMP6 at MOI of 5×10⁴ vg per AAV and imaged in the ImageXpress Micro (Molecular Devices) at 480 nm. AAV2.7m8 packaged with CMV-WT human melanopsin or CMV-SEQ ID NO: 19 successfully generated light response in HEK293T. The light response of SEQ ID NO: 19 was greater and faster than that of WT human melanopsin.

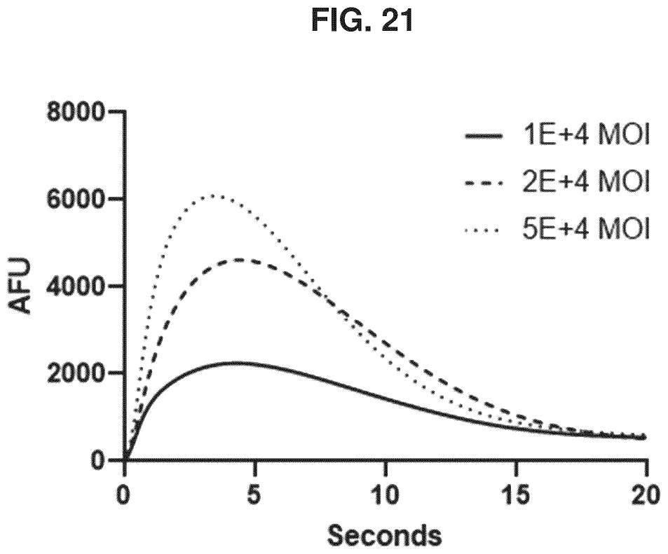

FIG. 21 shows the results of experiments that were performed to determine the amplitude of calcium light responses of HEK293T cells transduced with different MOIs of AAV2.7m8-SEQ ID NO: 19. Less AAV transduced resulted in a lower maximum amplification. The amplitude of the light response varied with the dose of AAV2.7m8-SEQ ID NO: 19 applied.

Although the foregoing disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

1. A melanopsin variant comprising no more than amino acids 1-425 of a wild type human melanopsin set forth in SEQ ID NO: 1, wherein amplitude/conductance and/or off kinetics of the melanopsin variant are greater than the amplitude/conductance and/or faster than the off kinetics of the wild type human melanopsin.

2. The melanopsin variant of claim 1, comprising the sequence set forth in any one of SEQ ID NOs: 2, 3, 4, 82, 83, and 84 or a variant thereof that comprises one or more amino acid substitutions.

3. The melanopsin variant of claim 2, wherein the variant comprises one or more amino acid substitution(s) at P10, T83, T129, Q135, S183, Y212, E215, M226, Y382, S384, R386, and/or R390, wherein amino acid position(s) are relative to the wild type human melanopsin set forth in SEQ ID NO: 1.

4. The melanopsin variant of claim 3, wherein the one or more substitution mutation(s) are selected from the group consisting of: P10F, T83L, T129S, Q135N, S183A, Y212F or Y212A, E215S, M226S or M226T, Y382E or Y382D, S384D, R386A, and R390A or R390D.

5. The melanopsin variant of claim 4, wherein the melanopsin variant comprises the P10F substitution.

6. The melanopsin variant of claim 5, wherein the melanopsin variant comprises SEQ ID NO: 5.

7. The melanopsin variant of claim 4, wherein the melanopsin variant comprises the T83L substitution.

8. The melanopsin variant of claim 7, wherein the melanopsin variant comprises SEQ ID NO: 6.

9. The melanopsin variant of claim 4, wherein the melanopsin variant comprises the T129S substitution.

10. The melanopsin variant of claim 9, wherein the melanopsin variant comprises SEQ ID NO: 7.

11. The melanopsin variant of claim 4, comprising the Q135N substitution.

12. The melanopsin variant of claim 11, wherein the melanopsin variant comprises SEQ ID NO: 8 or 9.

13. The melanopsin variant of claim 4, comprising the S183A substitution.

14. The melanopsin variant of claim 13, wherein the melanopsin variant comprises any one of SEQ ID NOs: 10-12.

15. The melanopsin variant of claim 4, comprising the Y212F substitution.

16. The melanopsin variant of claim 15, wherein the melanopsin variant comprises SEQ ID NO: 13.

17. The melanopsin variant of claim 4, comprising the M226S or the M226T substitution.

18. The melanopsin variant of claim 17, wherein the melanopsin variant comprises SEQ ID NO: 14 or 15.

19. The melanopsin variant of claim 4, wherein the melanopsin variant comprises the Y382E or the Y382D substitution.

20. The melanopsin variant of claim 19, wherein the melanopsin variant comprises SEQ ID NO: 16.

21. The melanopsin variant of claim 4, wherein the melanopsin variant comprises the S384D substitution.

22. The melanopsin variant of claim 21, wherein the melanopsin variant comprises SEQ ID NO: 17.

23. The melanopsin variant of claim 4, wherein the melanopsin variant comprises the R386A substitution.

24. The melanopsin variant of claim 23, wherein the melanopsin variant comprises SEQ ID NO: 18 or 19.

25. The melanopsin variant of claim 4, wherein the melanopsin variant comprises the R390A or the R390D substitution.

26. The melanopsin variant of claim 25, wherein the melanopsin variant comprises any one of SEQ ID NOs: 20-23.

27. A melanopsin variant comprising at least amino acids 1-377 of a wild type human melanopsin set forth in SEQ ID NO: 1 fused to a c-terminal domain (CTD) of a heterologous G protein-coupled receptor (GPCR) or a CTD variant thereof, wherein amplitude/conductance and/or off kinetics of the melanopsin variant are greater than the amplitude/conductance and/or faster than the off kinetics of the wild type human melanopsin.

28-34. (canceled)

35. A melanopsin variant comprising one or more amino acid substitutions at P10, T83, T129, Q135, S183, Y212, E215, M226, Y382, S384, R386, and R390, wherein amino acid positions are relative to a wild type human melanopsin set forth in SEQ ID NO: 1, and wherein amplitude/conductance and/or off kinetics of the melanopsin variant are greater than the amplitude/conductance and/or faster the off kinetics of the wild type human melanopsin.

36-38. (canceled)

39. A nucleic acid comprising a polynucleotide sequence that encodes the melanopsin variant of claim 1.

40. The nucleic acid of claim 39, operably linked to a retinal cell-specific promoter, wherein the retinal cell-specific promoter is selected from the group consisting of: human synapsin (hSyn), SNCG, NEFH, NEFL, 4×grm6, and grm6.

41-42. (canceled)

43. The nucleic acid of claim 39, further comprising one or more enhancer sequences, intron sequences, leader sequences, Kozak sequences, poly A sequences, stuffer sequences, and/or inverted terminal repeat (ITR) sequences.

44. A recombinant virion comprising:
(a) a capsid protein and
(b) the nucleic acid of claim 39.

45. The recombinant virion of claim 44, wherein the capsid protein is selected from: AAV2-7m8, AAV2, AAV2-4YF, AAV9, AAV9-7m8, R100 and LSV1.

46. A host cell comprising the nucleic acid of claim 39.

47. The host cell of claim 46, further comprising one or more of:
(i) a polynucleotide encoding a capsid protein;
(ii) a polynucleotide encoding a rep protein; and
(iii) AAV helper functions

48. A method for producing a recombinant virion, comprising:
(a) culturing the host cell of claim 46 under conditions to produce the recombinant virion,
(b) recovering the recombinant virion produced by the host cell, and
(c) purifying the recombinant virion.

49. (canceled)

50. A pharmaceutical composition comprising the recombinant virion of claim 44 and a pharmaceutically acceptable excipient.

51. A method of restoring or enhancing visual function in a subject, comprising administering the pharmaceutical composition of claim 50 to the eye of the subject.

52. The method of claim 51, wherein the administration comprises an intraocular injection, a subretinal injection, a suprachoroidal injection, or an intravitreal injection.

53. The method of claim 51, wherein:
i) the subject has an ocular disease or disorder selected from the group consisting of: retinitis pigmentosa, macular degeneration, retinoschisis, Leber's Congenital Amaurosis, diabetic retinopathy, geographic atrophy, choroideremia, cone dystrophy, and cone-rod dystrophy; or
ii) the subject has experienced retinal detachment or photoreceptor loss due to ocular disease, infection, trauma, injury, impact to the head, acute light damage, UV light damage, laser damage, or chemical damage.

54. (canceled)


SEQUENCE LISTING (165 sequences)

SEQ ID NO: 1 - protein, 478 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVGWTHMEAAAVWGAAQQANGRSLYGQGLEDLEAKAPPRPQGHEAETPGKTKGLIPSQDPRM

SEQ ID NO: 2 - protein, 420 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVGW

SEQ ID NO: 3 - protein, 419 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 4 - protein, 413 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLG

SEQ ID NO: 5 - protein, 419 aa, synthetic construct
MNPPSGPRVFPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 6 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLLGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 7 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFSSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 8 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKNWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 9 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFSSSLYKNWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 10 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVAAKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 11 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKNWLFGETGCEFYAFCGALFGISSMITLTAIALDRYLVITRPLATFGVAAKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 12 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFSSSLYKNWLFGETGCEFYAFCGALFGISSMITLTAIALDRYLVITRPLATFGVAAKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 13 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAFVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 14 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYTSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 15 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAFVPEGLLTSCSWDYTSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVG

SEQ ID NO: 16 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPDPSYRSTHRSTLTSHTSNLSWISIRRRQESLGSESEVG

SEQ ID NO: 17 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPDYRSTHRSTLTSHTSNLSWISIRRRQESLGSESEVG

SEQ ID NO: 18 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYASTHRSTLTSHTSNLSWISIRRRQESLGSESEVG

SEQ ID NO: 19 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVAAKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPDYASTHRSTLTSHTSNLSWISIRRRQESLGSESEVG

SEQ ID NO: 20 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHDSTLTSHTSNLSWISIRRRQESLGSESEVG

SEQ ID NO: 21 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPDPSYASTHDSTLTSHTSNLSWISIRRRQESLGSESEVG

SEQ ID NO: 22 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVAAKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPDPSYASTHDSTLTSHTSNLSWISIRRRQESLGSESEVG

SEQ ID NO: 23 - protein, 419 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFSSSLYKQWLFGETGCEFYAFCGALFGISSMITLTAIALDRYLVITRPLATFGVAAKRRAAFVLLGVWLYALAWSLPPFFGWSAYVPEGLLTSCSWDYTSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPDPSYASTHDSTLTSHTSNLSWISIRRRQESLGSESEVG

SEQ ID NO: 24-26 - skipped (3 entries)

SEQ ID NO: 27 - protein, 14 aa, synthetic construct
TEVSTVSSTQVGPN

SEQ ID NO: 28 - protein, 42 aa, synthetic construct
NKQFQACIMKMVCGKAMTDESDTCSSQKTEVSTVSSTQVGPN

SEQ ID NO: 29 - protein, 27 aa, synthetic construct
AMTDESDTCSSQKTEVSTVSSTQVGPN

SEQ ID NO: 30 - protein, 15 aa, synthetic construct
DAQSQATASEAESKA

SEQ ID NO: 31 - skipped

SEQ ID NO: 32 - protein, 12 aa, synthetic construct
TEVSSVSSVSPA

SEQ ID NO: 33 - protein, 37 aa, synthetic construct
QFRNCILQLFGKKVDDGSELSSASKTEVSSVSSVSPA

SEQ ID NO: 34-36 - skipped (3 entries)

SEQ ID NO: 37 - protein, 417 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRERNVRSAFTTSTVVRMHVGDGKSSSAASRSSSLVNLWKRR

SEQ ID NO: 38 - protein, 420 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRERNVRSAFTTSTVVRMHVGDGKSSSAASRSSSLVNLWKRR

SEQ ID NO: 39 - protein, 394 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRTEVSTVSSTQVGPN

SEQ ID NO: 40 - protein, 422 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRNKQFQACIMKMVCGKAMTDESDTCSSQKTEVSTVSSTQVGPN

SEQ ID NO: 41 - protein, 407 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRAMTDESDTCSSQKTEVSTVSSTQVGPN

SEQ ID NO: 42 - protein, 396 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPDAQSQATASEAESKA

SEQ ID NO: 43 - protein, 414 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRQKFRVTFLKIWNDHFRCQPAPPIDEDLPEERPDD

SEQ ID NO: 44 - protein, 392 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRTEVSSVSSVSPA

SEQ ID NO: 45 - protein, 417 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRQFRNCILQLFGKKVDDGSELSSASKTEVSSVSSVSPA

SEQ ID NO: 46 - protein, 10 aa, synthetic construct
LALGETTRPA

SEQ ID NO: 47 - protein, 10 aa, synthetic construct
LANETITRPA

SEQ ID NO: 48 - protein, 10 aa, synthetic construct
LAKAGQANNA

SEQ ID NO: 49 - protein, 10 aa, synthetic construct
LAKDPKTTNA

SEQ ID NO: 50 - protein, 7 aa, synthetic construct
KDTDTTR

SEQ ID NO: 51 - protein, 7 aa, synthetic construct
RAGGSVG

SEQ ID NO: 52 - protein, 7 aa, synthetic construct
AVDTTKF

SEQ ID NO: 53 - protein, 7 aa, synthetic construct
STGKVPN

SEQ ID NO: 54 - protein, 10 aa, synthetic construct
LAKDTDTTRA

SEQ ID NO: 55 - protein, 10 aa, synthetic construct
LARAGGSVGA

SEQ ID NO: 56 - protein, 10 aa, synthetic construct
LAAVDTTKFA

SEQ ID NO: 57 - protein, 10 aa, synthetic construct
LASTGKVPNA

SEQ ID NO: 58 - protein, 7 aa, synthetic construct
HKFKSGD

SEQ ID NO: 59 - protein, 10 aa, synthetic construct
LAHKFKSGDA

SEQ ID NO: 60 - protein, 10 aa, synthetic construct
LAHKFKSGDA

SEQ ID NO: 61 - protein, 401 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSKKVDDGSELSSASKTEVSSVSSVSPA

SEQ ID NO: 62 - protein, 401 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRVDDGSELSSASKTEVSSVSSVSPA

SEQ ID NO: 63 - protein, 392 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITNKQFRNCMLTTICCGKNPLGDDEASATVSKTETSQVAPA

SEQ ID NO: 64 - protein, 394 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRLALKEKCPCCVFGKVDDGKSSDAQSQATASEAESKA

SEQ ID NO: 65 - protein, 394 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLVFGKVDDGKSSDAQSQATASEAESKA

SEQ ID NO: 66 - protein, 399 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRLALKEKCPCILQLFGKKVDDGSELSSASKTEVSSVSSVSPA

SEQ ID NO: 67 - protein, 399 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRLALKEKCPCCVFGKVKKVDDGSELSSASKTEVSSVSSVSPA

SEQ ID NO: 68 - protein, 468 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQMNPPSGPRVPPSPTQEPSCMATPAPPSWWDSSQSSISSLGRLPSISPTAPGTWAAAWVPLPTVDVPDHAHAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRLALKEKCPCCVFGKVDDGKSSELSSASKTEVSSVSSVSPA

SEQ ID NO: 69 - protein, 395 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITNKQFQACIMKMVCGKAMTDESDTCSSQKTEVSTVSSTQVGPN

SEQ ID NO: 70 - protein, 402 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCIMKMVCGKAMTDESDTCSSQKTEVSTVSSTQVGPN

SEQ ID NO: 71 - protein, 403 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSKAMTDESDTCSSQKTEVSTVSSTQVGPN

SEQ ID NO: 72 - protein, 403 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRMTDESDTCSSQKTEVSTVSSTQVGPN

SEQ ID NO: 73 - protein, 410 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRVTFLKIWNDHFRCQPAPPIDEDLPEERPDD

SEQ ID NO: 74 - protein, 404 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRVDDGSELSSASKTEVSSVSSVSPA

SEQ ID NO: 75 - protein, 466 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 76 - protein, 392 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPQFRNCILQLFGKKVDDGSELSSASKTEVSSVSSVSPA

SEQ ID NO: 77 - protein, 414 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRQFRNCILQLFGKKVDDGSELSSASKTEVSSVSSVSPA

SEQ ID NO: 78 - protein, 442 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 79 - protein, 464 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 80 - protein, 389 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPQKFRVTFLKIWNDHFRCQPAPPIDEDLPEERPDD

SEQ ID NO: 81 - protein, 411 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRQKFRVTFLKIWNDHFRCQPAPPIDEDLPEERPDD

SEQ ID NO: 82 - protein, 400 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNL

SEQ ID NO: 83 - protein, 412 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISISEQIDNO

SEQ ID NO: 84 - protein, 432 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVGWTHMEASEQIDNO

SEQ ID NO: 85 - protein, 10 aa, synthetic construct
LAHQDTTKNS

SEQ ID NO: 86 - protein, 10 aa, synthetic construct
LALGETTRAA

SEQ ID NO: 87 - protein, 10 aa, synthetic construct
LAHQDTTRPA

SEQ ID NO: 88 - protein, 10 aa, synthetic construct
LARQDTTKNA

SEQ ID NO: 89 - protein, 10 aa, synthetic construct
LAHQDSTKNA

SEQ ID NO: 90 - protein, 10 aa, synthetic construct
LAHQDATKNA

SEQ ID NO: 91 - protein, 10 aa, synthetic construct
LAHQDTTKPA

SEQ ID NO: 92 - protein, 10 aa, synthetic construct
IALSETTRPA

SEQ ID NO: 93 - protein, 10 aa, synthetic construct
LAHQDTTKKC

SEQ ID NO: 94 - protein, 10 aa, synthetic construct
LALGEATRPA

SEQ ID NO: 95 - protein, 10 aa, synthetic construct
LALGETTRTA

SEQ ID NO: 96 - protein, 10 aa, synthetic construct
LALSEATRPA

SEQ ID NO: 97 - protein, 10 aa, synthetic construct
LAKDETKNSA

SEQ ID NO: 98 - protein, 10 aa, synthetic construct
LALGETTKPA

SEQ ID NO: 99 - protein, 10 aa, synthetic construct
LAHQATTKNA

SEQ ID NO: 100 - protein, 16 aa, synthetic construct
LQRGNRQTTTADVNTQ

SEQ ID NO: 101 - protein, 16 aa, synthetic construct
LQRGNRQATTADVNTL

SEQ ID NO: 102 - protein, 16 aa, synthetic construct
LQRGNRQATTEDVNTQ

SEQ ID NO: 103 - protein, 16 aa, synthetic construct
LQRGNRQAATEDVNTQ

SEQ ID NO: 104 - protein, 16 aa, synthetic construct
LQRGNRQAATADVNSL

SEQ ID NO: 105 - protein, 16 aa, synthetic construct
LQRGNRQAATADVNKL

SEQ ID NO: 106 - protein, 16 aa, synthetic construct
LQRGVRVPSVLEVNGQ

SEQ ID NO: 107 - protein, 16 aa, synthetic construct
LQRGNRQAATADVNIL

SEQ ID NO: 108 - protein, 16 aa, synthetic construct
LQRGKRQATTADVNTQ

SEQ ID NO: 109 - protein, 16 aa, synthetic construct
LHRGNRQAATADVNTL

SEQ ID NO: 110 - protein, 19 aa, synthetic construct
SRTNTPSGTTTQPTLQFSQ

SEQ ID NO: 111 - protein, 19 aa, synthetic construct
SKTDTPSGTTTQSRLQFSQ

SEQ ID NO: 112 - protein, 19 aa, synthetic construct
SRTDTPSETTTQSRLQFSQ

SEQ ID NO: 113 - protein, 19 aa, synthetic construct
SRTNSPSGTTTQSSLQFSQ

SEQ ID NO: 114 - protein, 19 aa, synthetic construct
SRTDIPSGTTTQSRLQFSQ

SEQ ID NO: 115 - protein, 7 aa, synthetic construct
HQDTTKN

SEQ ID NO: 116 - protein, 7 aa, synthetic construct
LGETTRA

SEQ ID NO: 117 - protein, 7 aa, synthetic construct
HQDTTRP

SEQ ID NO: 118 - protein, 7 aa, synthetic construct
RQDTTKN

SEQ ID NO: 119 - protein, 7 aa, synthetic construct
HQDSTKN

SEQ ID NO: 120 - protein, 7 aa, synthetic construct
HQDATKN

SEQ ID NO: 121 - protein, 7 aa, synthetic construct
HQDTTKP

SEQ ID NO: 122 - protein, 7 aa, synthetic construct
LSETTRP

SEQ ID NO: 123 - protein, 7 aa, synthetic construct
HQDTTKK

SEQ ID NO: 124 - protein, 7 aa, synthetic construct
LGEATRP

SEQ ID NO: 125 - protein, 7 aa, synthetic construct
LGETTRT

SEQ ID NO: 126 - protein, 7 aa, synthetic construct
LSEATRP

SEQ ID NO: 127 - protein, 7 aa, synthetic construct
KDETKNS

SEQ ID NO: 128 - protein, 7 aa, synthetic construct
LGETTKP

SEQ ID NO: 129 - protein, 7 aa, synthetic construct
HQATTKN

SEQ ID NO: 130 - protein, 7 aa, synthetic construct
ISDQTKH

SEQ ID NO: 131 - protein, 7 aa, synthetic construct
QADTTKN

SEQ ID NO: 132 - protein, 7 aa, synthetic construct
ASDSTKA

SEQ ID NO: 133 - protein, 7 aa, synthetic construct
NQDYTKT

SEQ ID NO: 134 - protein, 7 aa, synthetic construct
HDITKNI

SEQ ID NO: 135 - protein, 7 aa, synthetic construct
HPDTTKN

SEQ ID NO: 136 - protein, 7 aa, synthetic construct
HQDTTKN

SEQ ID NO: 137 - protein, 7 aa, synthetic construct
NKITNKD

SEQ ID NO: 138 - protein, 7 aa, synthetic construct
ISNENEH

SEQ ID NO: 139 - protein, 7 aa, synthetic construct
QANANEN

SEQ ID NO: 140 - protein, 7 aa, synthetic construct
GKSKVID

SEQ ID NO: 141 - protein, 7 aa, synthetic construct
TNRTSPD

SEQ ID NO: 142 - protein, 7 aa, synthetic construct
PNSTHGS

SEQ ID NO: 143 - protein, 7 aa, synthetic construct
KDRAPST

SEQ ID NO: 144 - protein, 9 aa, synthetic construct
LAQADTKNA

SEQ ID NO: 145 - protein, 10 aa, synthetic construct
LAISDQTKHA

SEQ ID NO: 146 - protein, 10 aa, synthetic construct
LGISDQTKHA

SEQ ID NO: 147 - protein, 10 aa, synthetic construct
LAASDSTKAA

SEQ ID NO: 148 - protein, 10 aa, synthetic construct
LANQDYTKTA

SEQ ID NO: 149 - protein, 10 aa, synthetic construct
LAHDITKNIA

SEQ ID NO: 150 - protein, 10 aa, synthetic construct
LAHPDTTKNA

SEQ ID NO: 151 - protein, 10 aa, synthetic construct
LAHQDTTKNA

SEQ ID NO: 152 - protein, 10 aa, synthetic construct
LANKTTNKDA

SEQ ID NO: 153 - protein, 10 aa, synthetic construct
LPISNENEHA

SEQ ID NO: 154 - protein, 10 aa, synthetic construct
LPQANANENA

SEQ ID NO: 155 - protein, 10 aa, synthetic construct
LAGKSKVIDA

SEQ ID NO: 156 - protein, 10 aa, synthetic construct
LATNRTSPDA

SEQ ID NO: 157 - protein, 10 aa, synthetic construct
LAPNSTHGSA

SEQ ID NO: 158 - protein, 10 aa, synthetic construct
LAKDRAPSTA

SEQ ID NO: 159 - protein, 395 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPERNVRSAFTTSTVVRMHVGDGKSSSAASRSSSLVNLWKRR

SEQ ID NO: 160 - protein, 384 aa, synthetic construct
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPARGRTPPSLGPQDESCTTASSSLAKDTSS

SEQ ID NO: 161 - protein, 5 aa, synthetic construct
GGGGS

SEQ ID NO: 162 - protein, 6 aa, synthetic construct
GGGGSS

SEQ ID NO: 163 - protein, 7 aa, synthetic construct
GGGGSSS

SEQ ID NO: 164 - protein, 10 aa, synthetic construct
GGGGSGGGGS

SEQ ID NO: 165 - protein, 15 aa, synthetic construct
GGGGSGGGGSGGGGS